(19) United States
(12) Patent Application Publication
(10) Pub. No.: US 2011/0082376 A1
(43) Pub. Date: Apr. 7, 2011

(54) PHYSIOLOGICAL BLOOD PRESSURE WAVEFORM COMPRESSION IN AN ACOUSTIC CHANNEL

(76) Inventors: Paul J. Huelskamp, St. Paul, MN (US); Joseph E. Bange, Eagan, MN (US); Jon N. Peterson, Mahtomedi, MN (US)

(21) Appl. No.: 12/854,703

(22) Filed: Aug. 11, 2010

Related U.S. Application Data
(60) Provisional application No. 61/248,703, filed on Oct. 5, 2009.

(51) Int. Cl. A61B 5/0215; G06F 19/00; G06F 11/07

(52) U.S. Cl. 600/486; 702/19; 714/746; 714/E11.023

(57) ABSTRACT

Methods, systems and devices for processing blood pressure measurements are disclosed. An illustrative method includes transmitting a command to an implantable medical device including a blood pressure sensor. The method includes receiving a response from the implantable medical device, the response indicating that the device initiated the sensing of blood pressure measurements. The method includes receiving one or more data packets from the implantable medical device. The one or more data packets can include a base pressure measurement that is representative of a starting point of a blood pressure waveform. The one or more data packets further include a delta value measurement representative of a difference between another blood pressure measurement and the base pressure measurement, or a difference between a current measurement and one or more previous measurements. Additionally, the method includes generating a pressure waveform from the one or more data packets.

### CROSS-REFERENCE TO RELATED APPLICATIONS

This application claims the benefit under 35 U.S.C. §119 of U.S. Provisional Application No. 61/248,703, filed Oct. 5, 2009, entitled “Physiological Blood Pressure Waveform Compression In An Acoustic Channel,” which is incorporated herein by reference in its entirety for all purposes.

### TECHNICAL FIELD

The present invention relates to the sampling and transmission of sensor data. More specifically, the present invention pertains to the sampling of blood pressure measurements and the transmission of samples as data packets over a wireless channel.

### BACKGROUND

Blood pressure measurements are used by hospitals and physicians to ascertain a patient's vital statistics. However, the traditional method of measuring systemic blood pressure, such as using a mechanical cuff along with a stethoscope, can be time-consuming and inaccurate. Accordingly, digitized measurements of blood pressure have been developed to improve accuracy in measuring blood pressure. Additionally, transmission of blood pressure data through a wireless channel may alleviate the problem of requiring the physician or nurse to be physically present with the patient.

Transmission of blood pressure data through a wireless channel, however, may present problems. For example, blood pressure data transmitted over a wireless channel can sometimes require additional payload bits to facilitate transmission of the blood pressure data, which increases the amount of data sent over the wireless channel. As an example, synchronization bits may be included in the data packets to help detect incoming data packets. As another example, the channel may be very noisy, causing corruption of the blood pressure data. Accordingly, parity bits for error detection and correction may be inserted in the data packet. Thus, the sampled data along with these additional payload bits may not fit in a wireless channel having limited bandwidth.

### SUMMARY

Embodiments include a method of processing blood pressure measurements transmitted from an implantable medical device to a receiving device. In Example 1, the method includes transmitting a command to an implantable medical device including a blood pressure sensor, the command prompting the device to sense blood pressure measurements within the body. The method further includes receiving a response from the implantable medical device, where the response indicates that the device initiated the sensing of blood pressure measurements. The method further includes receiving one or more data packets from the implantable medical device. The one or more data packets include a base pressure measurement that is representative of a starting point of a blood pressure waveform. The one or more data packets also include a measurement representative of a difference between another blood pressure measurement and a previous pressure measurement. The method also includes generating a pressure waveform from the one or more data packets.

In Example 2, the method according to Example 1, wherein the command includes at least a measuring interval indicating a time interval for sampling the blood pressure waveform, a marker interval indicating a time interval for sending a marker, and a sampling rate indicating a time interval between sampling sensed blood pressure measurements of the blood pressure waveform.

In Example 3, the method according to any of Examples 1-2, further comprising: performing error correction on the one or more received packets using one or more parity bits included in each packet of the received one or more data packets, wherein the one or more parity bits included in each packet of the received one or more data packets includes at least one row parity bit and at least one column parity bit; and detecting an error when at least one bit in the one or more received packets does not correspond to the at least one row parity bit and the at least one column parity bit.

In Example 4, the method according to any of Examples 1-3, further comprising: synchronizing the one or more received packets using a marker included in the one or more received packets, the marker having a unique value non-representative of the blood pressure measurements, wherein a first packet received after the marker is received is identified as a first portion of the base pressure measurement, and a second packet received after the first packet is identified as a second portion of the base pressure measurement.

In Example 5, the method according to any of Examples 1-4, further comprising: reconstructing a blood pressure waveform from the received one or more data packets, wherein, for each data point on the blood pressure waveform, the measurement representative of a difference between the another blood pressure measurement and the base pressure measurement is added to the base pressure measurement.

In Example 6, the method according to any of Examples 1-5, wherein the measurement representative of the difference is encoded using an encoding range including a range of discrete values.

In Example 7, the method according to any of Examples 1-6, wherein the one or more data packets include at least a sync bit, a predetermined number of data bits, a row parity bit, and a column parity bit.

In Example 8, an implantable medical device configured to sample a blood pressure waveform, the device comprising: a response module configured to receive a command from a communicating device, the command including parameters for sampling a blood pressure waveform, the parameters specifying at least a sampling rate and a resolution; the response module configured to transmit a response packet to the communicating device in response to the command; a pressure measurement module configured to sense the blood pressure waveform; an ADC module configured to obtain a blood pressure sample by sampling the blood pressure waveform at the sampling rate; a resolution conversion module configured to convert the blood pressure sample to the resolution; a delta module configured to determine a difference measurement between the converted blood pressure sample and a previous blood pressure sample; an encoding module configured to encode the determined difference measurement using an encoding range including a range of discrete values; a packet module configured to generate a data packet including the difference measurement; and the packet module configured to transmit the data packet to the communicating device.

In Example 9, the device of Example 8, wherein the packet module is further configured to transmit a marker to the device in accordance with a marker interval, the marker interval included in the command, the marker having a unique value non-representative of the blood pressure measurement; the packet module is further configured to transmit a first packet after transmission of the marker, the first packet including a first portion of a base pressure measurement; and the packet module is further configured to transmit a second packet after transmission of the first packet, the second packet including a second portion of the base pressure measurement.

In Example 10, the device of any of Examples 8-9, further comprising: a parity module configured to determine a row parity bit by counting a number of ones included in the data packet; and the parity module configured to determine a column parity bit of a packet set transmitted prior to the transmission of the data packet, wherein the packet module is further configured to prepare the data packet by inserting the row parity bit and column parity bit in the data packet.

In Example 11, the device of any of Examples 8-10, wherein the encoding module is further configured to adjust the encoding range using a range of the blood pressure values of the sampled blood pressure waveform.

In Example 12, the device of any of Examples 8-11, wherein: the encoding module is further configured to determine an overflow result when the difference measurement exceeds a maximum range of the encoded range; and the packet module is further configured to transmit the overflow result in another data packet.

In Example 13, the device of any of Examples 8-12, wherein the data packet includes at least a sync bit, a number of data bits, a row parity bit, and a column parity bit.

In Example 14, a system for sampling a blood pressure waveform, the system comprising: an external device communicable with an implantable medical device, the external device including: a command module configured to transmit a command to the implantable medical device, the command including parameters for sampling a blood pressure waveform; and the implantable medical device including: a response module configured to transmit a response packet to a second device in response to the command; a pressure measurement module configured to obtain a blood pressure measurement by sampling the blood pressure waveform at a sampling rate; a resolution conversion module configured to convert the blood pressure measurement to a resolution; a delta module configured to determine a difference measurement between the converted sampled blood pressure waveform and a previous sample; an encoding module configured to encode the determined difference measurement using an encoding range including a range of discrete values; and a packet module configured to prepare the determined difference measurement as a data packet; wherein the packet module is configured to transmit the data packet to the external device.

In Example 15, the system according to Example 14, wherein: the packet module is further configured to transmit a marker to the external device in accordance with a marker interval, the marker interval included in the command, the marker having a unique value non-representative of the blood pressure measurement; the packet module is further configured to transmit a first packet after transmission of the marker, the first packet including a first portion of a base pressure measurement; and the packet module is further configured to transmit a second packet after transmission of the marker, the second packet including a second portion of the base pressure measurement.

In Example 16, the system according to any of examples 14-15, wherein the implantable device further comprises: a parity module configured to determine a row parity bit by counting a number of ones included in the data packet; the parity module is configured to determine a column parity bit of a packet set transmitted prior to the transmission of the data packet; and wherein the packet module is further configured to prepare the data packet by inserting the row parity bit and column parity bit in the data packet.

In Example 17, the system according to any of Examples 14-16, wherein the external device further includes: an error detection module configured to detect one or more errors in the data packet using at least the row parity bit and the column parity bit; and an error correction module configured to correct the one or more errors in the data packet.

In Example 18, the system according to any of Examples 14-17, wherein the encoding module is further configured to adjust the encoding range using a range of the sampled blood pressure waveform.

In Example 19, the system according to any of Examples 14-18, wherein the packet module is further configured to determine an overflow result when the determined difference exceeds a maximum range of the encoded range and transmit the overflow result in another data packet.

In Example 20, the system according to any of Examples 14-19, wherein the external device further includes: a waveform reconstruction module configured to reconstruct the blood pressure waveform by adding the encoded difference included in the data packet to a base pressure measurement.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an illustrative system employing a remote implantable medical device (IMD) located within the body of a patient;

FIG. 2 is block diagram showing several illustrative components of the remote IMD of FIG. 1;

FIG. 3 is a block diagram showing several illustrative components of a communicating device such as the handheld device;

FIG. 4 illustrates an example system;

FIG. 5 illustrates another example system;

FIG. 6 illustrates an example send data command;

FIG. 7A illustrates an example response byte header;

FIG. 7B illustrates an example response byte;

FIG. 8 illustrates an example blood pressure waveform;

FIG. 9 illustrates an example format for sampling a blood pressure waveform at 40 Hz;

FIG. 10 illustrates an example encoding format of a delta value;

FIG. 11 illustrates an example pressure stream (at 1 mmHg resolution) with both uncompressed and non-linear compression modes;

FIGS. 12A-12C illustrate input values to the output values for the non-linear compression.

FIG. 13 illustrates example data;

FIG. 14 illustrates example data including row and parity bits;

FIG. 15 illustrates example data with a single bit error;

FIG. 16 illustrates an example process for sampling a blood pressure waveform and transmitting samples as data packets;

FIG. 17 illustrates an example process for receiving data packets and performing error detection and correction;

FIG. 18 illustrates an example process for reconstructing a waveform; and

FIG. 19 is a schematic diagram of an example computing device.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

A patient's blood pressure may be measured via an implantable medical device (IMD). When a patient's blood pressure measurement is taken, the patient's blood pressure is sensed by the IMD and reproduced as a waveform. Accordingly, in embodiments, the blood pressure waveform may be sampled, where the samples are transmitted over a wireless channel and read at a handheld device and/or pulse generator (PG). In some embodiments, a base pressure measurement is synchronized with a marker. As an example, after a marker is sent from the IMD to the handheld device, the next two values that are sent are an upper portion of the base pressure measurement and a lower portion of the base pressure measurement. After the base pressure measurement is sent, a difference value between the pressure measurements is sent in a data packet instead of the actual pressure measurement in order to conserve bandwidth. As an example, a 10 bit data sampled at 40 Hz may not fit in a 500 bits/sec channel along with additional payload bits. Accordingly, by sending a 6-bit difference value instead of an actual pressure measurement, bandwidth may be conserved.

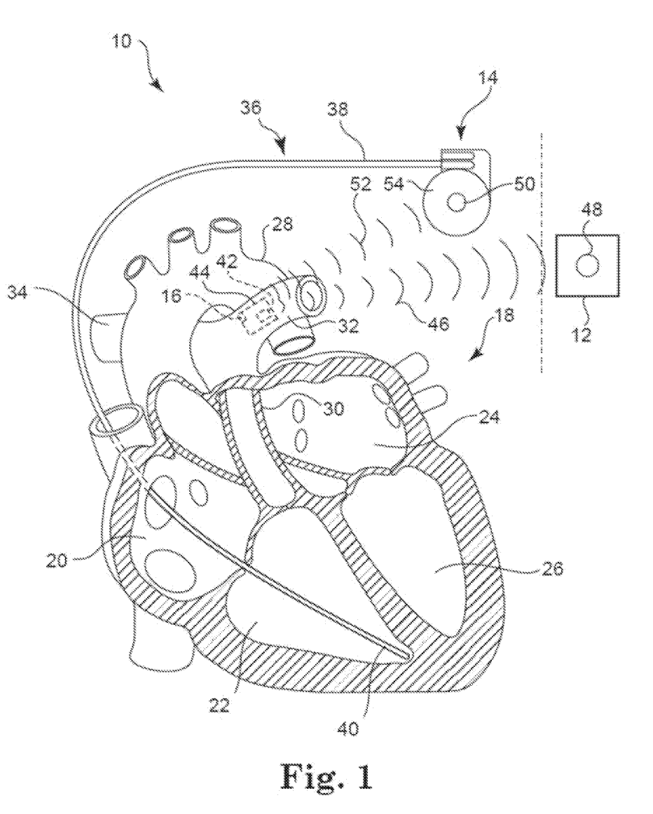

FIG. 1 is a schematic view of an illustrative system 10 employing a remote implantable medical device (IMD) located within the body of a patient. The system 10, illustratively a cardiac rhythm management system for providing cardiac rhythm management or cardiac disease management, includes an handheld device 12 (e.g., an external communicator, reader, or programmer), a pulse generator 14 implanted within the body, and at least one remote IMD 16 implanted deeply within the patient's body such as in one of the atria or ventricles of the patient's heart 18, or in one of the blood vessels leading into or from the heart 18. The heart 18 includes a right atrium 20, a right ventricle 22, a left atrium 24, a left ventricle 26, and an aorta 28. The right ventricle 22 leads to the main pulmonary artery 30 and the branches 32, 34 of the main pulmonary artery 30.

In the illustrative system 10 depicted, the pulse generator 14 is coupled to a lead 36 deployed in the patient's heart 18. The pulse generator 14 can be implanted subcutaneously within the body, typically at a location such as in the patient's chest or abdomen, although other implantation locations are possible. A proximal portion 38 of the lead 36 can be coupled to or formed integrally with the pulse generator 14. A distal portion 40 of the lead 36, in turn, can be implanted at a desired location within the heart 18 such as the right ventricle 22, as shown. Although the illustrative system 10 depicts only a single lead 36 inserted into the patient's heart 18, in other embodiments the system 10 may include multiple leads so as to electrically stimulate other areas of the heart 18. In some embodiments, for example, the distal portion of a second lead (not shown) may be implanted in the right atrium 20. In addition, or in lieu, another lead may be implanted in the left side of the heart 18 (e.g., in the coronary veins) to stimulate the left side of the heart 18. Other types of leads such as epicardial leads may also be utilized in addition to, or in lieu of, the lead 36 depicted in FIG. 1.

During operation, the lead 36 is configured to convey electrical signals between the heart 18 and the pulse generator 14. For example, in those embodiments where the pulse generator 14 is a pacemaker, the lead 36 can be utilized to deliver electrical therapeutic stimulus for pacing the heart 18. In those embodiments where the pulse generator 14 is an implantable cardiac defibrillator, the lead 36 can be utilized to deliver electric shocks to the heart 18 in response to an event such as ventricular fibrillation. In some embodiments, the pulse generator 14 includes both pacing and defibrillation capabilities.

The remote IMD 16 can be configured to perform one or more designated functions, including the sensing of one or more physiologic parameters within the body. Example physiologic parameters that can be measured using the remote IMD 16 can include, but are not limited to, blood pressure, blood flow, and temperature. Various electrical, chemical, magnetic, and/or sound properties may also be sensed within the body via the remote IMD 16.

In the embodiment of FIG. 1, the remote IMD 16 comprises a pressure sensor implanted at a location deep within the body such as in the main pulmonary artery 30 or a branch 32, 34 of the main pulmonary artery 30 (e.g., in the right or left pulmonary artery). An example of a pressure sensor suitable for use in sensing blood pressure in a pulmonary artery is described in U.S. Pat. No. 6,764,446, entitled “Implantable Pressure Sensors and Methods for Making and Using Them,” which is incorporated herein by reference in its entirety for all purposes. In use, the remote IMD 16 can be used to aid in the prediction of decompensation of a heart failure patient and/or to aid in optimizing cardiac resynchronization therapy via the pulse generator 14 by monitoring blood pressure within the body. In some embodiments, the remote IMD 16 can be configured to sense, detect, measure, calculate, and/or derive other associated parameters such as flow rate, maximum and minimum pressure, peak-to-peak pressure, rms pressure, and/or pressure rate change.

The remote IMD 16 may be implanted in other regions of the patient's vasculature, in other body lumens, or in other areas of the body, and may comprise any type of chronically implanted device adapted to deliver therapy and/or monitor biological and chemical parameters, properties, and functions. The remote IMD 16 can be tasked, either alone or with other implanted or external devices, to provide various therapies or diagnostics within the body. Although a single remote IMD 16 is depicted in FIG. 1, multiple such devices can be implanted at various locations within the body for sensing or monitoring physiologic parameters and/or providing therapy at multiple regions within the body.

An acoustic communication link may be established to permit wireless communications between the remote IMD 16 and the handheld device 12, between the remote IMD 16 and the pulse generator 14, and/or between the remote IMD 16 and one or more other devices located inside or outside of the body. In the illustrative system 10 of FIG. 1, for example, an ultrasonic transducer 42 disposed within the housing 44 of the remote IMD 16 is configured to transmit an ultrasound signal 46 towards the handheld device 12. An example ultrasonic transducer suitable for use with the remote IMD 16 for transmitting and receiving ultrasound signals is described in U.S. Pat. No. 6,140,740, entitled “Piezoelectric Transducer,” which is expressly incorporated herein by reference in its entirety for all purposes.

The handheld device 12 includes one or more ultrasonic transducers 48 configured to receive the ultrasound signal 46 and complete an acoustic link between the remote IMD 16 and the handheld device 12. In some cases, for example, the acoustic link established between the remote IMD 16 and the handheld device 12 can be used to wirelessly transmit sensor data, operational status information, and/or other information to the handheld device 12. An example telemetry system employing ultrasonic transducers is described in U.S. Pat. No. 7,024,248, entitled “Systems and Methods For Communicating With Implantable Devices,” which is incorporated herein by reference in its entirety for all purposes.

In some embodiments, the ultrasonic transducer(s) 48 for the handheld device 12 may transmit an ultrasound signal to the remote IMD 16 to prompt the IMD 16 to perform a desired operation. In one embodiment, for example, the handheld device 12 may transmit an acoustic wake-up command to the remote IMD 16, causing the IMD 16 to activate from an initial, low-power state for conserving power usage to an active, energized state for taking one or more sensor measurements and transmitting sensor data to the handheld device 12, to the pulse generator 14, and/or to another device located inside or outside of the body. In some embodiments, and as further discussed herein, the handheld device 12 may transmit an acoustic control signal that prompts the remote IMD 16 to wake up only a portion of the IMD 16 and transmit one or more ultrasonic pulses without activating the sensor circuitry within the IMD 16.

While the system 10 of FIG. 1 includes a remote IMD 16 that communicates with a handheld device 12, in other embodiments the remote IMD 16 communicates with other devices located inside or outside of the patient's body. As further shown in FIG. 1, for example, the remote IMD 16 may be in acoustic communication with the pulse generator 14, which can include one or more ultrasonic transducers 50 adapted to receive an ultrasound signal 52 transmitted by the remote IMD 16. In certain embodiments, the ultrasonic transducer(s) 50 are coupled to an interior portion of the can 54 that encloses the various components of the pulse generator 14. In other embodiments, the ultrasonic transducer(s) 50 are located outside of the can 54, on a header of the can 54, or are coupled to the pulse generator 14 through a feedthrough provided on the can 54.

Although the system 10 depicted in FIG. 1 shows an acoustic link between the remote IMD 16 and a handheld device 12, and/or between the IMD 16 and a pulse generator 14, in other embodiments an acoustic link can be established between the remote IMD 16 and another device implanted within the body. In some embodiments, for example, an acoustic link can be established between a primary IMD 16 and one or more secondary IMDs 16 implanted within the body.

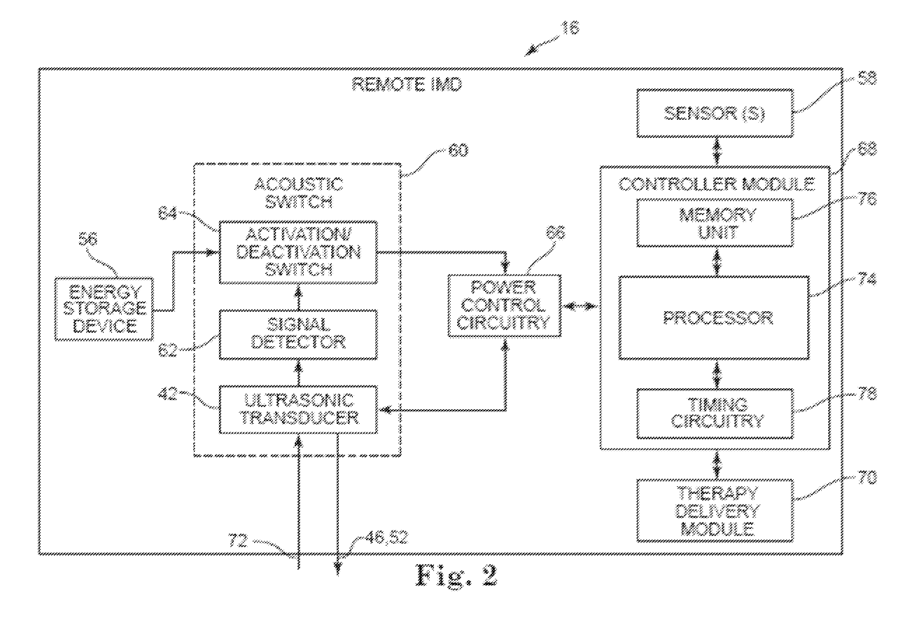

FIG. 2 is block diagram showing several illustrative components of the remote IMD 16 of FIG. 1. As shown in FIG. 2, the remote IMD 16 includes an energy storage device 56, a physiologic sensor 58, an acoustic switch 60 (including the acoustic transducer 42, a signal detector 62, and an activation/deactivation switch component 64), power control circuitry 66, and a controller module 68. The energy storage device 56 may be non-rechargeable or rechargeable, and supplies power to the physiologic sensor 58, the acoustic switch 60, the power control circuitry 66, and the controller module 68. The power control circuitry 66 is operatively connected to the acoustic switch 60, and is used to regulate the supply of power from the energy storage device 56 to the controller module 68.

The physiologic sensor 58 performs functions related to the sensing of one or more physiologic parameters within the body. In certain embodiments, for example, the physiologic sensor 58 comprises a pressure sensor adapted to measure blood pressure in the body. In one embodiment, the remote IMD 16 is implanted in a pulmonary artery of the patient, and the physiologic sensor 58 is adapted to sense blood pressure within the artery. In other embodiments, the physiologic sensor 58 is adapted to generate a signal related to other sensed physiologic parameters including, but not limited to, temperature, electrical impedance, pH, blood flow, and glucose level. In certain embodiments, the remote IMD 16 may also include a therapy delivery module 70 that performs one or more therapeutic functions (e.g., cardiac pacing or drug delivery) within the body in addition to, or in lieu of, the one or more sensing functions provided by the physiologic sensor 58.

The ultrasonic transducer 42 for the remote IMD 16 may include one or more piezoelectric transducer elements configured to transmit and receive ultrasound signals. In a reception mode of operation, the ultrasonic transducer 42 can be configured to receive a control signal 72 transmitted from the handheld device 12 and/or the pulse generator 14, which is fed to the controller module 68 when the remote IMD 16 is in an active state. In a transmit mode of operation, the ultrasonic transducer 42, or another ultrasonic transducer coupled to the remote IMD 16, is configured to transmit an ultrasound signal 46, 52 to the handheld device 12, to the pulse generator 14, and/or to another device located inside or outside of the body. The transmitted ultrasound signal 46, 52 can include sensor data obtained from the physiologic sensor 58, information relating to the status or operation of the remote IMD 16 (e.g., power status, communication mode status, error correction information, etc.), as well as other information relating to the operation of the remote IMD 16.

The sensor data obtained by the physiologic sensor 58 and transmitted via the ultrasound signal 46, 52 may be encoded via on-off keying, phase-shift keying, frequency-shift keying, amplitude-shift keying, pulse code modulation, frequency modulation, amplitude modulation, or other suitable modulation techniques used in telemetry protocols. In on-off keying, for example, digitized sensor data is transmitted acoustically within a modulated carrier ultrasound signal 46, 52. The presence or absence of the carrier ultrasound signal 46, 52 is detected by the handheld device 12 or pulse generator 14 as either a binary “1” or “0,” respectively.

The signal detector 62 is configured to generate an activation trigger signal to activate the remote IMD 16 via the activation/deactivation switch component 64. The activation trigger signal is generated by the signal detector 62 when the electrical signal generated by the ultrasonic transducer 42 exceeds a specific voltage threshold.

In response to the generation of the activation trigger signal by the signal detector 62, the switch component 64 is actuated to allow current to flow from the energy storage device 56 to the controller module 68, thereby placing the remote IMD 16 in the active state. The switch component 64 can also be actuated to prevent current from flowing to the controller module 68, thereby placing the remote IMD 16 in the standby or sleep state. Further details regarding the general construction and function of acoustic switches are disclosed in U.S. Pat. No. 6,628,989, entitled “Acoustic Switch And Apparatus And Methods For Using Acoustic Switches Within The Body,” which is expressly incorporated herein by reference in its entirety for all purposes. In other embodiments, the remote IMD 16 can include an antenna or inductive coil that receives an RF or inductive signal from the handheld device 12 or pulse generator 14 to activate or deactivate the remote IMD 16 within the body.

In embodiments, the controller module 68 includes a processor 74 such as a microprocessor or microcontroller coupled to a memory unit 76 that includes operating instructions and/or software for the remote IMD 16. In alternative embodiments, the controller module 68 is implemented as a program in software. The memory unit 76 can include volatile memory and nonvolatile memory. In some embodiments, nonvolatile memory can store code that includes bootstrap functions and device recovery operations, such as microprocessor reset. The nonvolatile memory may also include calibration data and parameter data in some embodiments. The volatile memory can include diagnostic and/or microprocessor-executable code, operating parameters, status data, and/or other data.

The controller module 68 can also include an oscillator or other timing circuitry 78 which directs the timing of activities to be performed by the remote IMD 16 once awoken from its low-power or sleep state. For example, the timing circuitry 78 can be used for timing the physiologic measurements taken by the physiologic sensor 58 and to generate timing markers to be associated with those measurements. The timing circuitry 78 may also be used for modulating the ultrasound signal 46, 52.

The controller module 68, including the processor 74, can be configured as a digital signal processor (DSP), a field programmable gate array (FPGA), an application specific integrated circuit (ASIC)-compatible device, and/or any other hardware components or software modules for processing, analyzing, storing data, and controlling the operation of the remote IMD 16. Processor 74 executes instructions stored in the memory 76 or in other components such as, for example, the physiologic sensor(s) 58 or therapy delivery module 70 and/or other components or modules that may be present. In general, processor 74 executes instructions that cause the processor 74 to control or facilitate the functions of the remote IMD 16 and/or components of the remote IMD 16.

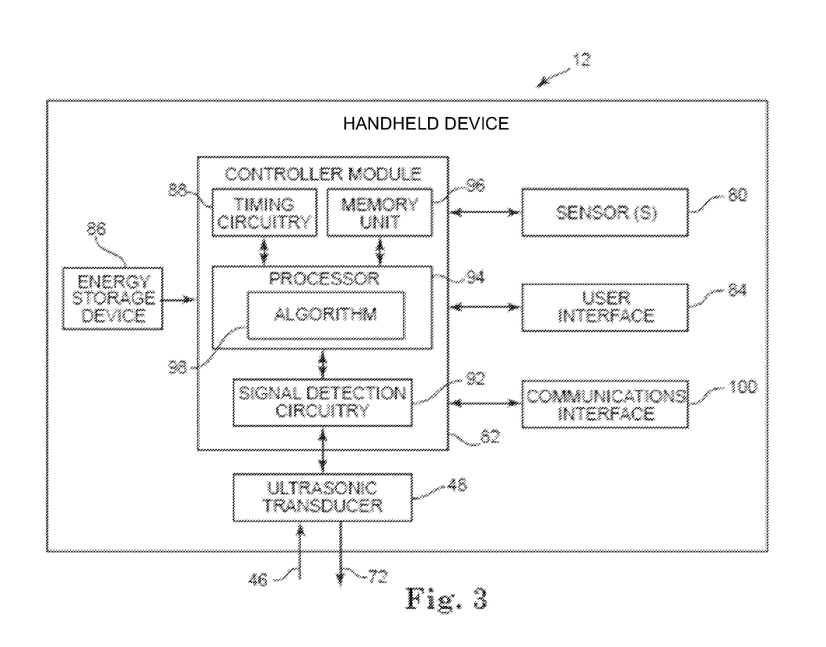

FIG. 3 is a block diagram showing several illustrative components of a communicating device such as the handheld device 12 of FIG. 1. As shown in FIG. 3, the handheld device 12 includes an ultrasonic transducer 48, one or more sensors 80, a controller module 82, a user interface 84, and an energy storage device 86. In some embodiments, the handheld device 12 is a handheld device. In other embodiments, the handheld device 12 is attached to a portion of the patient's body such as the patient's arm, neck, chest, thigh, or knee. The handheld device 12 can use any type of attachment mechanism, such as a strap, a patch, a belt, or any other means for coupling the hand held device 12 to the patient's body.

The one or more sensors 80 can include a biosensor that generates a signal in response to a sensed physiologic parameter, or an environmental sensor that generates a signal in response to a sensed environmental parameter. In one embodiment, for example, the sensor 80 comprises a barometric pressure sensor configured to measure barometric pressure for use in calibrating pressure data sensed by the remote IMD 16. The handheld device 12 may include one or more additional sensors such as an ECG electrode sensor, a systemic blood pressure sensor, a posture sensor, a global positioning system (GPS) sensor, an activity sensor, a temperature sensor, a timer, and/or an oximeter.

The ultrasonic transducer 48 for the handheld device 12 can be configured to both transmit and receive ultrasound signals to and from the remote IMD 16. In other embodiments, the handheld device 12 includes at least one transducer configured for receiving ultrasound signals from the remote IMD 16 and at least one transducer configured for transmitting ultrasound signals to the remote IMD 16. The ultrasonic transducer 48 generates an electrical signal proportional to the magnitude of acoustic energy received by the transducer 48, which is then conveyed to the controller module 82 as an electrical waveform. In similar fashion, the ultrasonic transducer 48 generates an ultrasound signal proportional to the magnitude of the electrical energy generated by the controller module 82.

The controller module 82 includes circuitry for activating or controlling the sensor 80 and for receiving signals from the sensor 80. In some embodiments, the controller module 82 may include an oscillator or other timing circuitry 88 for use in modulating the ultrasound signal transmitted to the remote IMD 16 and/or the pulse generator 14 via the ultrasonic transducer 48. In some embodiments, the controller module 82 further includes signal detection circuitry 92 for detecting ultrasound signals 46 received from the remote IMD 16 and/or the pulse generator 14 via the ultrasonic transducer 48.

The controller module 82 includes a processor 94 for analyzing, interpreting, and/or processing the received ultrasound signal 46, and a memory unit 96 for storing the processed information and/or commands for use internally. The memory unit 96 can include volatile memory and nonvolatile memory. In some embodiments, nonvolatile memory can store code that includes bootstrap functions and device recovery operations, such as microprocessor reset. The nonvolatile memory may also include calibration data and parameter data in some embodiments. The volatile memory can include diagnostic and/or microprocessor-executable code, operating parameters, status data, and/or other data.

The controller module 82, including the processor 94, can be configured as a digital signal processor (DSP), a field programmable gate array (FPGA), an application specific integrated circuit (ASIC)-compatible device, and/or any other hardware components or software modules for processing, analyzing, storing data, and controlling the operation of the handheld device 12. Processor 94 executes instructions stored in the memory unit 96 or in other components such as, for example, the sensor(s) 80, user interface 84, communications interface 100 and/or other components or modules that may be present. In general, processor 94 executes instructions that cause the processor 94 to control or facilitate the functions of the handheld device 12 and/or components of the handheld device 12.

In certain embodiments, a processor 94 can be configured to run an algorithm or routine that, in addition to decoding the sensor data from the ultrasound signal 46 and analyzing the sensor data, also analyzes the amplitude and timing characteristics of the received ultrasound signal 46 to determine one or more additional physiologic parameters within the body based on a direct measure of the signal 46 itself. In one embodiment, for example, the amplitude and timing characteristics of the ultrasound signal 46 received by the handheld device 12 can be analyzed to determine a second physiologic waveform such as respiration, which can be correlated with the pressure waveform data encoded and transmitted as part of the ultrasound signal 46. The pressure and respiration waveforms can be further analyzed together to determine precisely the end diastolic pressure occurring at end expiration.

The user interface 84 can include a screen or display panel for communicating information to a physician and/or to the patient. In certain embodiments, the user interface 84 can also be used to display other information such as any physiologic parameters sensed by the remote IMD 16 or the handheld device 12 and the power and operational status of the remote IMD 16. The user interface 84 can also display information regarding the characteristics of the ultrasound signal 46 received from the remote IMD 16, including, but not limited to the pressure of the ultrasound signal 46, the carrier frequency of the ultrasound signal 46, and the modulation format of the ultrasound signal 46 (e.g., on-off keying, phase-shift keying, frequency-shift keying, amplitude-shift keying, pulse code modulation, frequency modulation, amplitude modulation, etc.), and/or the presence of any communication errors that may have occurred in the transmission.

In some embodiments, the handheld device 12 can include a communications interface 100 for connecting the monitor 12 to the Internet, an intranet connection, to a patient management database, and/or to other wired or wireless means for downloading and/or uploading information and programs, debugging data, and upgrades. According to some embodiments, the handheld device 12 is capable of operating in two modes: a user mode that provides useful clinical information to the patient or a caregiver, and a diagnostic mode that provides information to an individual for calibrating and/or servicing the handheld device 12 or for changing one or more parameters of the remote IMD 16.

The term “module” refers broadly to a software, hardware, or firmware component (or any combination thereof). Modules are typically functional components that can generate useful data or other output using specified input(s). A module may or may not be self-contained. An application program (also called an “application”) may include one or more modules, and/or a module can include one or more application programs.

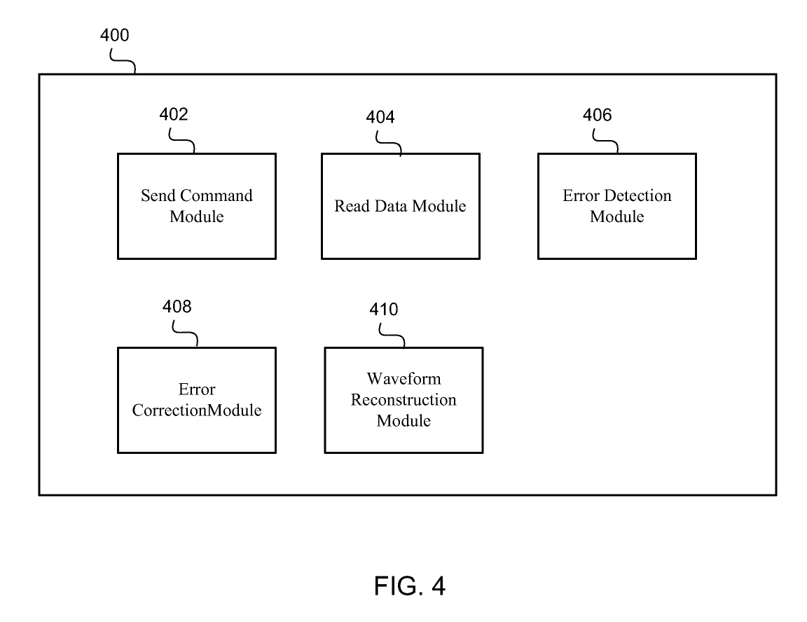

FIG. 4 illustrates an example system 400, including a send data command module 402, a read data module 404, an error detection module 406, an error correction module 408, and a waveform reconstruction module 410. In embodiments, the system 400 is implemented on the hand held device of FIG. 3.

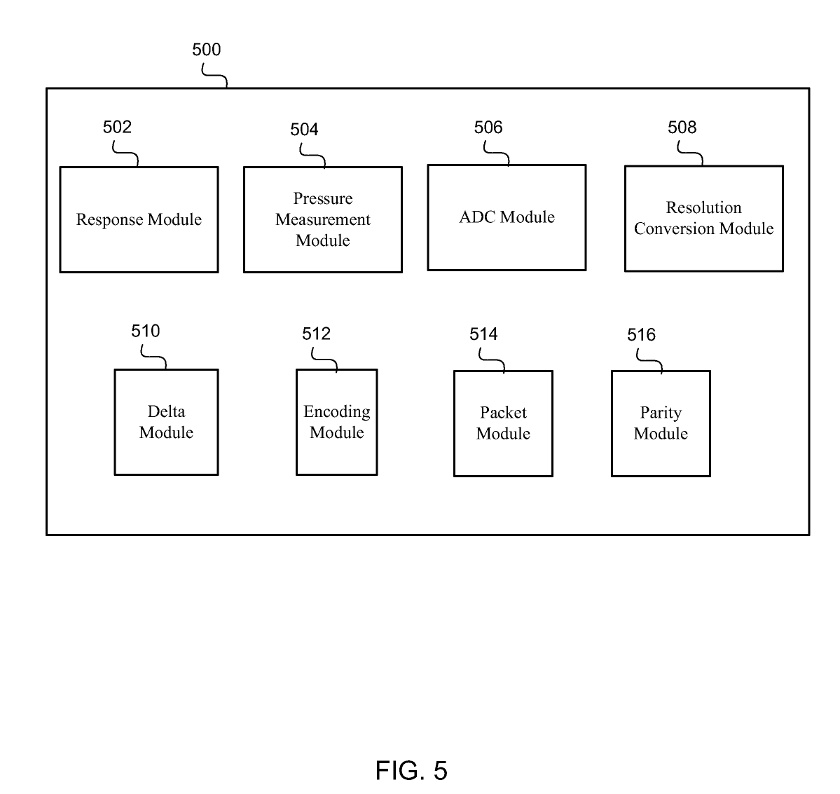

FIG. 5 illustrates a system 500 that includes a response module 502, a pressure measurement module 504, an analog to digital conversion (ADC) module 506, a resolution conversion module 508, a delta module 510, an encoding module 512, a packet module 514, and a parity module 516. In embodiments, the system 500 is implemented on the remote IMD 16 of FIG. 2.

In embodiments, one or modules within system 400 may be interchanged with one or modules within system 500. The operation of each of the modules illustrated in FIGS. 4 and 5 will be described in further detail below.

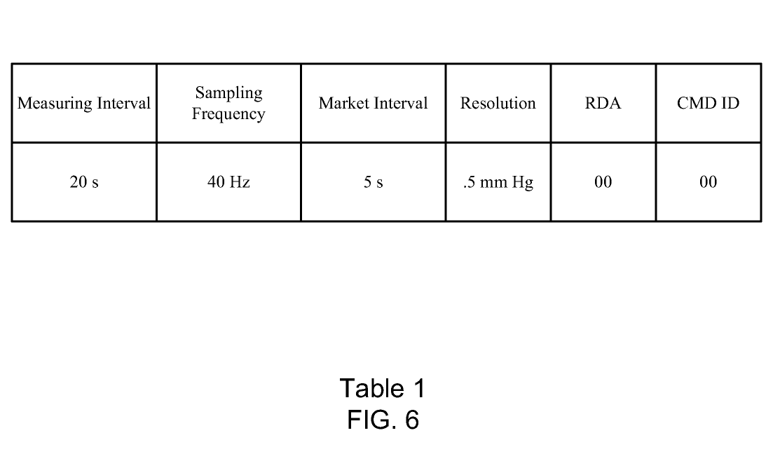

The sampling of a blood pressure waveform may be initiated when the hand held device 12 (FIG. 3) sends a send data command to the IMD 16 (FIG. 2). Table 1 of FIG. 6 illustrates an example send data command. In embodiments, the send data command module 402 (FIG. 4) transmits the send data command illustrated in Table 1. As an example, the send data command of Table 1 specifies a measuring interval of 20 seconds, a sampling frequency of 40 Hz, a marker interval of 5 seconds, a resolution of 0.5 mmHg, a relative device address (RDA) of 00, and a command (CMD) ID of 00. The measuring interval may indicate a specified or desired period of time for sampling a blood pressure waveform. The sampling frequency may indicate how often the blood pressure waveform is sampled. For example, with the sampling frequency of 40 Hz, the blood pressure waveform is sampled every 25 ms. The marker interval may specify the period of time between sending markers (e.g., a marker may be sent every 5 seconds). The resolution may specify the quality of the blood pressure sample such as the number of bits used in the blood pressure measurements. The RDA may specify the address of the device sending a command. For example, for a RDA of 00 with a command ID of 00 as illustrated in Table 1, the device address of the IMD 16 is 00, and the send data command is associated with a CMD ID of 00.

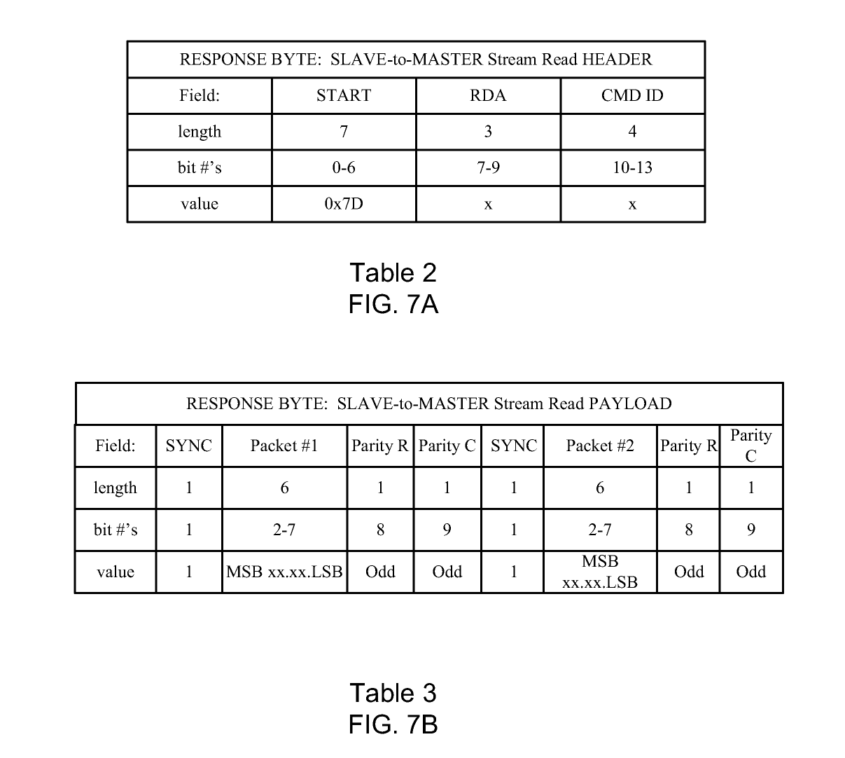

When the IMD 16 (FIG. 2) receives the send data command from the send data command module 402 (FIG. 4), the IMD 16 may send a response byte header to the hand held device 12 (FIG. 3) to indicate that the send data command was successfully received. In some embodiments, the response module 502 of system 500 (FIG. 5) transmits the response byte header. Table 2 of FIG. 7A illustrates an example response byte header. As an example, the response byte header may include a start field with a value of 0x7D, which may indicate a clock rate for synchronization between the IMD 16 and handheld device 12. The response byte header may also include an RDA and a CMD ID field, which may be similar to the RDA and CMD ID fields illustrated in Table 1. In embodiments, the response module 502 transmits the response byte header illustrated in Table 2 of FIG. 7A.

Upon issuing the response byte header, the packet module 514 may issue a response byte that includes one or more blood pressure samples in the form of packets. Table 3 of FIG. 7B illustrates an example response byte. In embodiments, the packet module 514 of system 500 transmits the response byte. As an example, each response byte may include two packets that are each 6 bits long. For example, the response byte of Table 3 includes a packet #1 and a packet #2. As illustrated in Table 3, the response byte includes 18 bits. The first bit before each packet may be a sync bit which synchronizes the handheld device 12 to sample the next six bits that are blood pressure samples. Additionally, the response byte may include two parity bits for each packet, where one parity bit may represent a row parity bit, and the other parity bit may represent a column parity bit. As discussed further herein, the row and/or column parity bits may be used to facilitate detection and correction of errors.

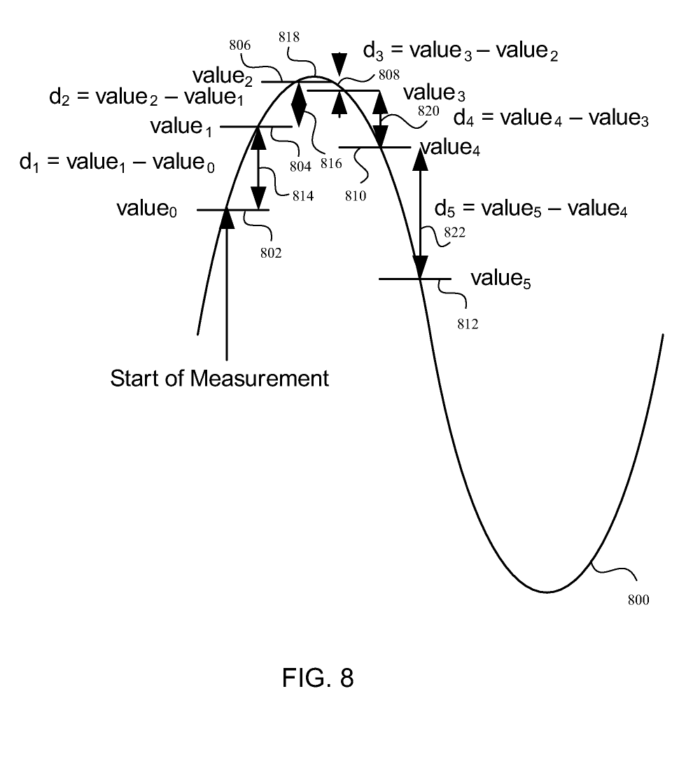

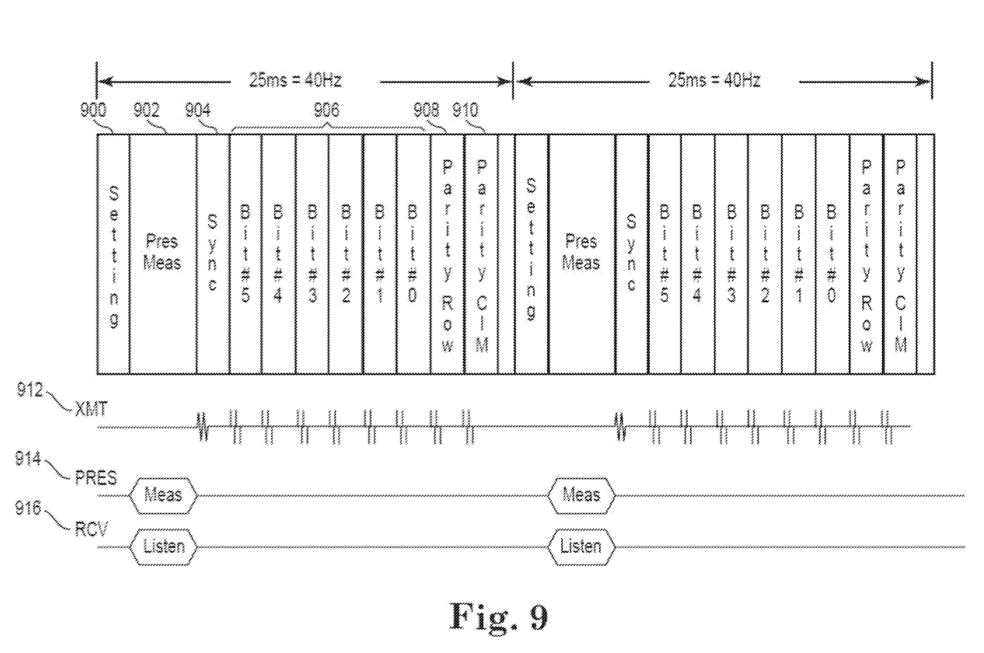

FIG. 8 illustrates an example blood pressure waveform 800. In embodiments, upon receiving the send data command from the hand held device 12, the pressure measurement module prompts the sensor 58 of IMD 16 to begin sensing the patient's blood pressure to create the blood pressure waveform 800. The blood pressure waveform 800 may be sampled, where the samples are transmitted from the IMD 16 to the hand held device 12 as packets. In embodiments, the ADC module 506 samples the blood pressure waveform 800. As illustrated in FIG. 8, the blood pressure waveform 800 includes sampled values Value₀ (802) to Value₅ (812) and delta values d₁ (814) to d₅ (822).

Value₀ (802) represents a base pressure measurement, which may be used as a starting point of the blood pressure waveform 800. Value₁ (804) represents the next sampled value after Value₀. Accordingly, a delta value d₁ represents the difference between Value₁ (804) and Value₀ (802). In embodiments, the base pressure measurement Value₀ (802) is an actual blood pressure measurement sampled from the blood pressure waveform 800 that is transmitted from the IMD 16 to the hand held device 12. In embodiments, after the base pressure measurement is transmitted, a delta value representative of a difference between the base pressure measurement and the next measurement is transmitted. That is, delta value d₁ is transmitted instead of Value₁. Subsequently, delta values representative of a difference between the previous pressure measurement and the next measurement are transmitted. By transmitting the delta value (e.g., d₁) instead of the actual blood pressure measurement (e.g., Value₁), bandwidth is conserved because fewer bits are used to transmit the delta value. Accordingly, delta values d₁ (814) to d₅ (816) may be transmitted instead of values Value₁ (804)-Value₅ (812) to conserve bandwidth. One aspect of a delta compression scheme is that once an un-correctable error is introduced, the data following the error is also corrupted. To minimize the impact this may cause to the data, markers can be periodically sent to give an absolute ADC value.

Below is a summary illustrating how several example delta values d₁ (814) to d₅ (816) may be calculated according to the blood pressure waveform illustrated in FIG. 8:

d₁=Value₁−Value₀

d₂=Value₂−Value₁

d₃=Value₃−Value₂

d₄=Value₄−Value₃

d₅=Value₅−Value₄

FIG. 9 illustrates an example format for sampling a blood pressure waveform at 40 Hz. As illustrated in FIG. 9, at a 40 Hz sampling rate, a sample pressure measurement is taken every 25 ms. Within the sampling period, there is a setting period 900 and a pressure measurement period 902. The setting period 900 may represent, for example, the period of time for the sensor 58 (FIG. 2) to finish sensing a previous blood pressure measurement. In some embodiments, the blood pressure measurement module 504 causes the sensor 58 to sense blood pressure measurements within the patient to generate a blood pressure waveform 800 during the pressure measurement period 902. After the pressure measurement period 902, a sync bit 904 and a six bit data packet 906 are transmitted. The sync bit 904 may be used to indicate the location of the next six bits that are the data packet. The six bit data packet 906 may be one of four types of packets. The first type is a marker value (e.g., all ones), that indicates that the next two six bit data packets will be combined to form a base pressure measurement. The second type is the most significant six bits (e.g., upper portion) of the base pressure measurement. The third type is the least significant six bits (e.g., lower portion) of the base pressure measurement. The fourth type is a delta value. After the six bit data packet is transmitted, a parity row bit 908 and a parity column bit 910 may be transmitted. The parity row bit 908 and parity column bit 910 may be used to facilitate in detecting and correcting errors. As illustrated in FIG. 9, once the current sampling period is over, the sampling period repeats itself until the measuring interval is completed. In embodiments, the packet module 514 prepares the samples as packets as illustrated in FIG. 9.

The transmit line (XMT) 912 may include a waveform representation of one or more bits. As an example, the XMT line 912 illustrates 9 pulses corresponding to the sync bit 904, the six bit data packet 906, the parity row bit 908, and the parity column bit 910. The pressure measurement section 914 illustrates a time period for performing a blood pressure measurement. The receive line (RCV) 916 illustrates a time period for listening for a prompt to start sensing a blood pressure measurement. In additional embodiments, a blood pressure waveform may be sampled at 25 Hz. When a blood pressure waveform is sampled at 25 Hz, a sample may be taken every 40 ms with a corresponding setting period, pressure measurement period between each sampling period. Additionally, a sync bit, data packet, parity row bit, and parity column bit may be prepared between each measuring period. In embodiments, when a blood pressure waveform is sampled at 25 Hz, there may be an additional delay period in between samples.

In embodiments, when the ADC module 506 starts sampling the blood pressure waveform 800 sensed by the blood pressure module 504, the ADC module 506 converts the samples from an analog to digital (hereinafter “ADC”) value. According to some embodiments, the first ADC value produced by the ADC module 506 is the base pressure measurement. As illustrated in FIG. 8, the base pressure measurement may be the starting point for the rest of the waveform 800. As an example, the base pressure measurement is sent out in the first three time slots. The first time slot may be a marker value, the second time slot may be the upper six bits of the base pressure measurement, and the third time slot may be the lower six bits of the base pressure measurement. After the base pressure measurement is transmitted, the subsequent packets transmitted may be the delta (e.g., change) from the previous value. In some embodiments, the delta value is six bits long, and the base pressure measurement is 12 bits long. In certain embodiments, the least significant bit (LSB) of the delta is determined by the resolution setting in the command.

In alternative embodiments, the base pressure measurement value sent in the three slot marker (e.g., marker packet, MSB packet, and LSB packet) corresponds to the measurement taken at the middle packet (MSB packet). This method allows better interpolation between the previous value from the base value and the next value after the base.

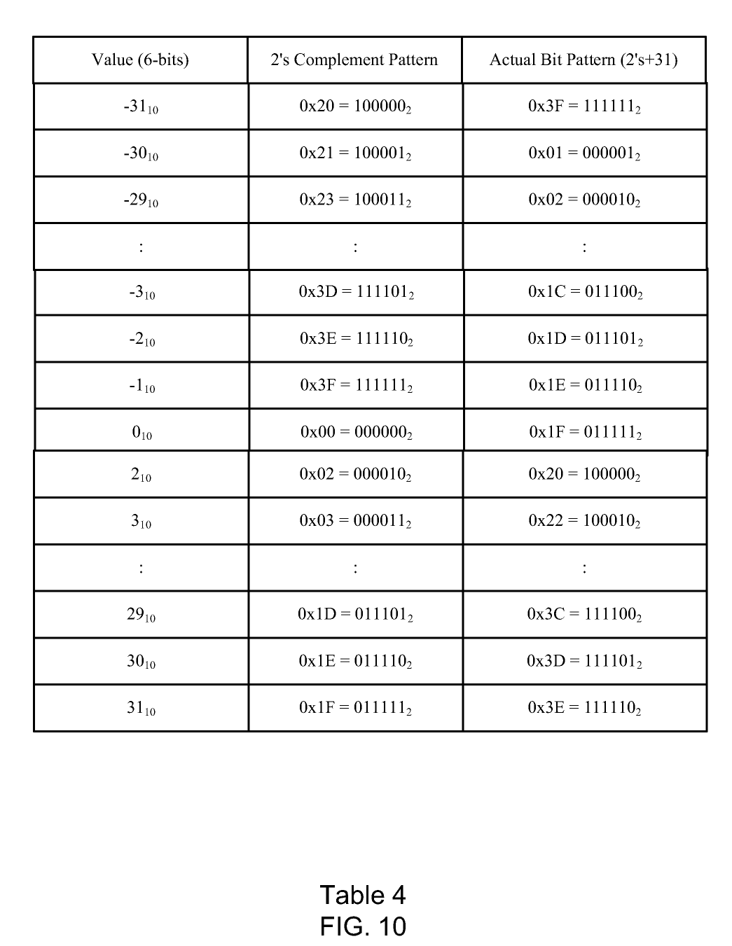

Table 4 of FIG. 10 illustrates an example encoding format for encoding the delta values generated from a sampled pressure waveform. As illustrated in Table 4, the values represented by the delta range from −31 to 31 with a difference of one integer between each value. As illustrated in Table 4, integer values between −31 and 31, such as ±1, ±2, ±3, ±4, ±5, etc., represent delta values having a unique encoded binary representation. In embodiments, the −31 and +31 values represent a minimum and maximum value, respectively, that may be transmitted for a delta. As illustrated in the third column of Table 4, the values −31 to 31 each have a unique encoded value. The actual bit pattern illustrated in the third column of Table 4 is derived by adding 31 to the corresponding 2's complement number. In embodiments, the 2's complement number may be derived by using the most significant bit (MSB) to indicate the sign of the number where a 1 at the MSB indicates a negative number, and a zero at the MSB indicates a positive number. As an example using a 4 bit binary number, the two's complement of a number may be obtained by subtracting 2ⁿ⁻¹ from the number. For example, 0111 is 7 where the 2's complement number 1111 is −1 (e.g., 7-8).

Additionally, as illustrated in Table 4, the encoding format includes a marker value (0x3F). Embodiments may use any desired encoding format. In embodiments, the encoding format illustrated in Table 4 accomplishes several goals. One goal of the encoding format is to use a single unique value to represent a marker value. This marker value may be inserted into the waveform periodically to allow the hand held device 12 to verify synchronization, or regain synchronization when data is lost. Particularly, when one or more deltas are lost during transmission, the waveform will not be accurately reconstructed with subsequent deltas. Accordingly, by transmitting a marker and a corresponding base pressure measurement, the hand held device 12 would have the ability to synchronize the deltas with the marker. In embodiments, the marker is a unique value that is not represented in the normal data stream. As an example, a value of six consecutive ones (0x3F) may be used as the marker, which is different from any other encoded value represented for values −31 to 31. In embodiments, the marker may be followed by a current 12 bit ADC value (6 bits at a time in each of the subsequent time slots). A second goal of the encoding format is to increase the number of ones transmitted to reduce the risk of losing synchronization or data dropout. That is, when there is an increase in the number of ones that the hand held device 12 receives, there is less risk of the clock on the hand held device 12 becoming out of sync. Additionally, during acoustic transmission, ones may be easier to detect than zeros. Accordingly, there may be less risk of losing data by increasing the number of ones that are transmitted. Additionally, the marker may provide synchronization on the column parity values since a marker may be set up to always occur in the first packet of a six packet field.

In alternative embodiments, a retroactive signal reconstruction method may be implemented. As an example, when one or more deltas are lost but synchronization is maintained (i.e., all subsequent deltas are good up to the next marker), reconstruction may be permitted by replacing the delta following the marker with a retroactive delta value. In embodiments, the retroactive delta would be the delta between the last data point prior to the marker, and the base pressure value following the marker. In further embodiments, the delta following the retroactive delta may be the difference between the base pressure value and the second value following the base pressure value.

In some embodiments, the encoding format illustrated in Table 4 of FIG. 10 is variable. As an example, after the delta module 510 determines the first few determined delta values, the encoding module 512 may adjust the encoding format to correspond to these first few values. For example, if the first few determined values are between −3 to 3, the encoding format may be changed so that there is more resolution between −3 and 3. Thus, the encoding format may be changed to provide delta values for ±0.5, ±1.5, ±2.5 and ±3, etc, depending in part on the first few determined values Accordingly, the encoding format may be adapted to each individual patient.

In other embodiments, the encoding format may be determined using a Huffman encoding method. As an example, in a Huffman encoding method, statistics on sample data is taken to determine which values are occurring the most. As an example, if the delta values from 0-3 occur more frequently, fewer bits may be used for these values. However, outside the range of 0-3, more bits are used for those values. Accordingly, by using a Huffman encoding method, bandwidth may be preserved because fewer bits are being transmitted for the values that occur the most. Embodiments may use any desired Huffman encoding method.

In additional embodiments, the encoded format in Table 4 of FIG. 10 is non-linear. In some embodiments, for example, the encoding format is based on a logarithmic scale where more resolution is provided between a smaller range of values, and less resolution is provided for the higher range of values. As an example, between the range of −10 and 10, there may be a unique binary representation for each increment of 0.25. As an example, the non-linear encoding format may specify a unique binary representation for 0.25, 0.5, 0.75, 1, 1.25, etc. Outside the range −10 to 10, a unique binary representation may be provided for each integer multiple of two. As an example, the non-linear encoding format may specify a unique binary representation that can be provided for 12, 14, 16, 18, 20, etc.

To further increase the resolution (and/or range) of the pressure sensor, an optional encoding mode may be implemented in some embodiments. This compression mode (e.g., non-linear data compression), allows the device to send twice the range of pressure deltas for a given 6-bit data word. For example, if 1 mmHg resolution is used, a single delta measurement could track a pressure change from −32 mmHg to +31 mmHg in the uncompressed mode. For the non-linear data compression, the same 1 mmHg resolution can track a pressure change from −60 mmHg to +63 mmHg.

As an example, for pressure values between −16 to +15, the data is mapped 1 to 1. That is, for every value on the input, there is an identical value on the output. For pressure values between −32 to −17, and +16 to +31, a 2:1 mapping is used. That is, a value of +16 or +17 is mapped to the same value, and values +18 to +19 are mapped to the same value (but different from the +16/17 value). For pressure values between −60 to −33, and +32 to +63, a 4:1 mapping is used such that a value of +32, +33, +34, or +35 will be mapped to the same value. Likewise +36, +37, +38, or +39 will be mapped to the same value (but different from the +32/33/34/35 value).

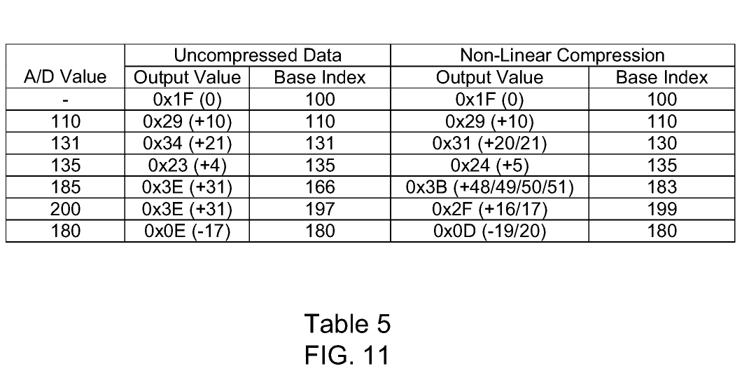

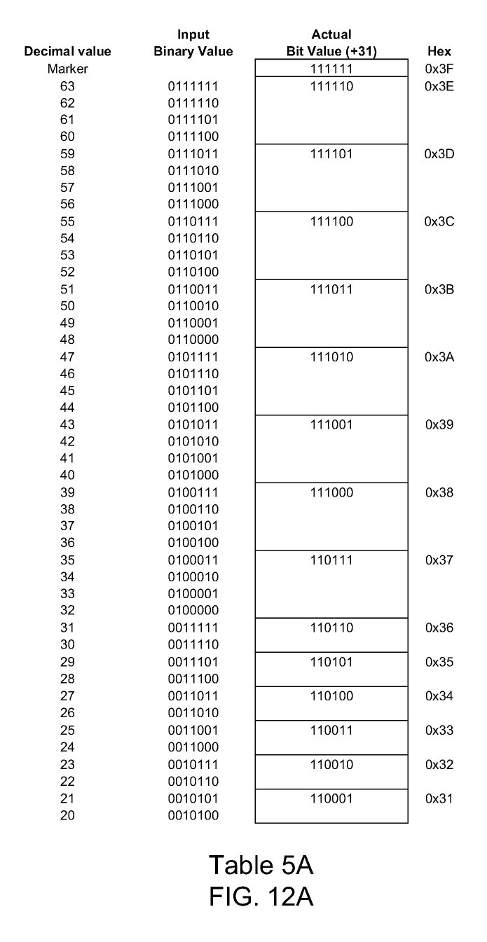

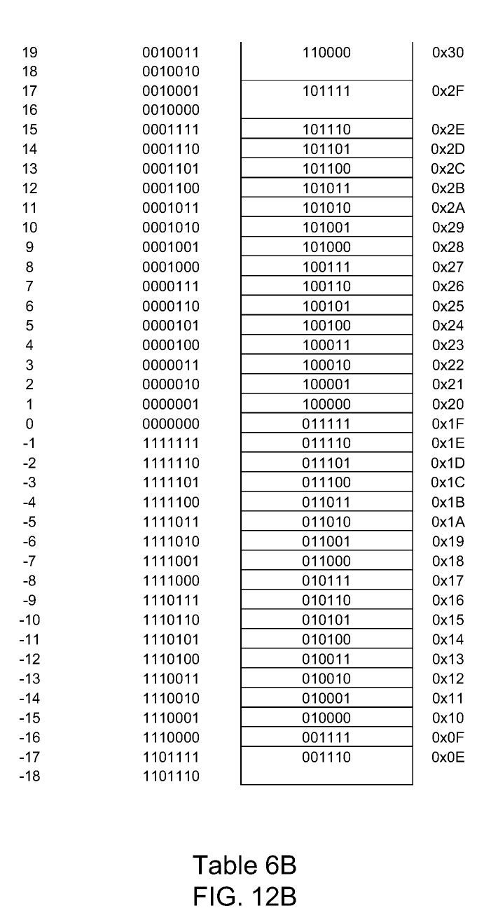

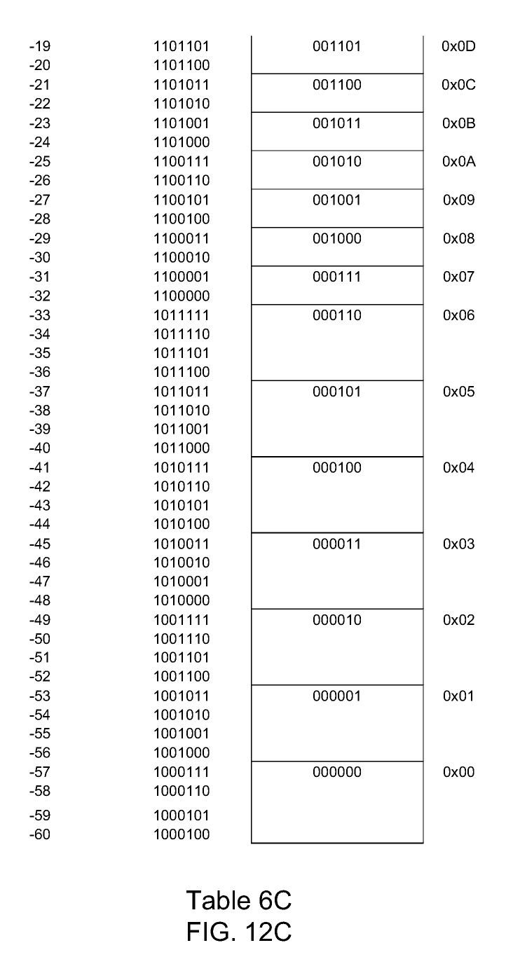

In embodiments, the exact pressure value is dependent on the resolution selected. For example, an output value of 0x27 represents a pressure delta of +8 mmHg at 1 mmHg resolution, or +4 mmHg at ½ mmHg resolution, or +2 mmHg at ¼ mmHg resolution, or +1 mmHg at ⅛ mmHg resolution. When a value is mapped into a compressed region (either 2:1 or 4:1), the pressure sensor module (PSM) uses the lowest value when determining the base value for the next pressure measurement. Any remainder is tacked onto the next value sent so that the over all accuracy is not impacted. An example pressure stream (at 1 mmHg resolution) is shown with both uncompressed and non-linear compression modes in Table 5 of FIG. 11. Tables 6A-6C of FIGS. 12A-12C illustrate input values to the output values for the non-linear compression.

According to some embodiments, the difference between the current pressure reading and the previous pressure reading may overflow the 6 bit range of the delta value. As an example, as illustrated in Table 4 of the FIG. 10, the range of delta values is from −31 to 31. Accordingly, when a difference between a current pressure value and a previous pressure value is, for example, 34, there is an overflow result since there is no unique binary representation for the number 34 in the encoding format. When this overflow result occurs, and in embodiments, the IMD 16 can be configured to send the maximum value (either +31 counts or −31 counts). The remainder of the difference will be tacked onto the next delta measurement. As an example, when the difference between the current pressure reading and the previous pressure reading is 34, there is an overflow result of three. Accordingly, a +31 is sent out for the current delta measurement and a +3 value is added to the next delta measurement. Thus, if the next delta measurement is 15, the next delta measurement would be 18.

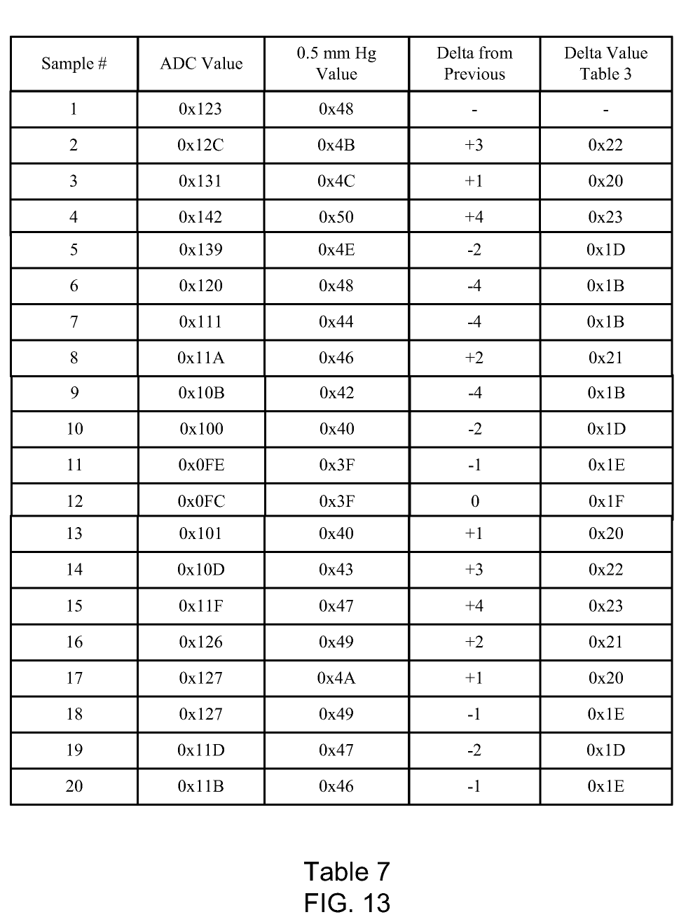

Table 7 of FIG. 13 illustrates example encoded pressure data with a measuring interval of 500 ms, a 40 Hz sampling rate, a 0.5 mmHg resolution, and a marker specified to be sent every 400 ms. As illustrated in Table 7 of FIG. 13, since there is a measuring interval of 500 ms with a sample occurring every 25 ms, 20 samples are taken during the measuring interval. The second column in Table 7 illustrates a 11 bit analog to digital value for each sample. As discussed above, and in some embodiments, the pressure measurement module 504 senses the blood pressure waveform 800 (FIG. 8), while the ADC module 506 samples the blood pressure waveform and converts the sample to a 12 bit digital value. Embodiments may use any desired analog to digital conversion method.

After the digital value is obtained, according to some embodiments, the digital value is converted to a specified resolution. As an example, the command illustrated in Table 1 of FIG. 6 specifies a resolution of 0.5 mm Hg. In embodiments, the resolution conversion module 508 (FIG. 5) converts the 12 bit digital sample (0.125 mmHg) to the proper resolution. To convert a digital value from 0.125 mmHg to 0.5 mmHg resolution, the digital value is shifted right by 2 bits. Accordingly, by shifting 2 bits from the value 0x12C (000100101100₂) to the right, the value is converted to 0x4B (0001001011₂). The fourth column of Table 7 illustrates a delta difference between the current value and the previous value. For example, the difference between sample two and sample one is 0x4B (75)-0x48 (72), which is equal to +3. As illustrated in Table 4 of FIG. 10, when a delta is +3, the encoded format is 0x22.

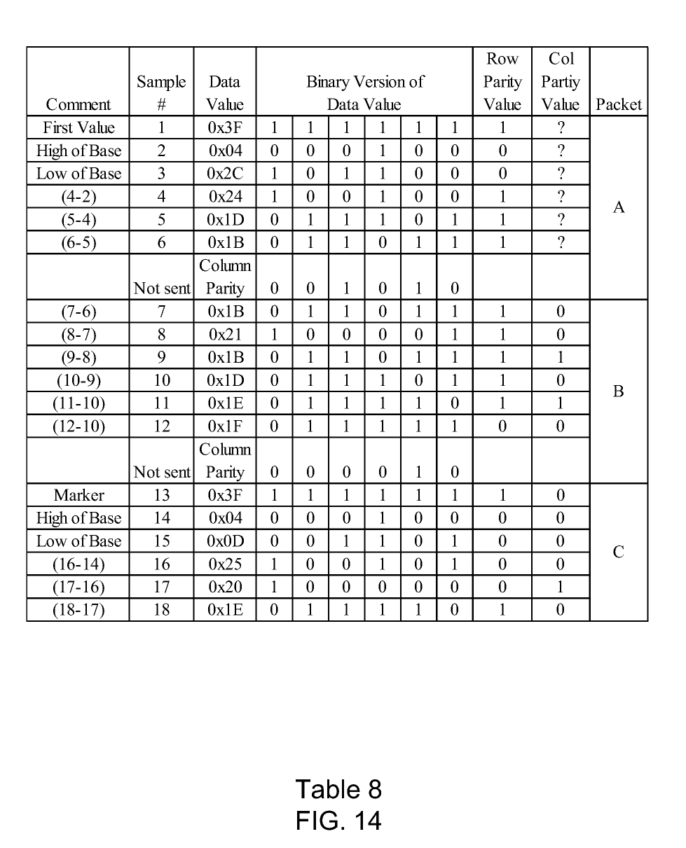

Table 8 of FIG. 14 illustrates several example data packet samples transmitted from the IMD 16 to the hand held device 12. The first column of Table 8 indicates the sample transmitted between the IMD 16 to the handheld device 12. For example, the first column indicates when a marker is transmitted or when the upper or lower portions of a base pressure measurement are transmitted. Additionally, the first column of Table 8 refers to the sample numbers from Table 7 that are subtracted from each other. The second column in Table 8 of FIG. 14 indicates a sample # for the sample transmitted between the IMD 16 and hand held device 12. The third column of Table 8 indicates the value transmitted, which may be a marker value (e.g., Sample #1 in Table 8), an upper portion of a base pressure measurement (e.g., Sample #2 in Table 8), a lower portion of a base pressure measurement (e.g., Sample #3 in Table 8), or a delta value (Sample #4 in Table 8).

According to some embodiments, the first value (e.g., sample #1 in Table 8) sent from the IMD 16 to the hand held device 12 is the marker (0x3F). The marker indicates to the hand held device 12 that a base value will be sent in the next two time slots (e.g., sample #'s 2 and 3 in Table 8). In some embodiments, according to the samples illustrated in Table 7 of FIG. 13, the base value is 0x12C. This value represents the middle value between the first three measured samples (i.e., Sample #'s 1, 2, and 3). The second value sent from the IMD 16 to the hand held device 12 is the upper 6 bits of 0x12C (000100101100₂), which is 0x04 (000100₂). The third value sent between IMD 16 and the hand held device 12 is the lower 6 bits of 0x12C, which is 0x2C (101100₂). The fourth value sent between the IMD 16 and the hand held device 12 is the first 6 bit delta value, which is 0x24.

The fourth value (e.g., sample #4 in Table 8) may be determined by subtracting 0x4B (sample #2 in Table 5) from 0x50 (sample #4 in Table 5). Accordingly, this result is +5. Next, according to the encoding format specified in Table 4 of FIG. 10, 31 is added to this result, which results in 36 in decimal and 0x24 in hexadecimal. As illustrated in this example, sample numbers 1 and 3 may be discarded to conserve bandwidth. The next delta value transmitted between the IMD 16 and the hand held device 12 is 0x1 (−1), 0x1B (−4), and 0x1B (−4), respectively. As illustrated in Table 8, a marker value (0x3F) is sent at sample #13 to re-synchronize a pressure value to an exact value and indicate that the next two packets include a base pressure measurement. The two data values following the marker, 0x04 and 0x0D, represent the upper 6 bits and the lower 6 bits of the 12 bit pressure value for Sample #14 (0x10D) (Table 8), respectively. In embodiments, the packet module 514 utilizes the ultrasonic transducer of the remote IMD 16 to transmit packets from the IMD 16 to the hand held device 12.

According to some embodiments, two parity values may be sent as part of the data packet. The first parity value may be a row parity bit. In embodiments, this row parity bit represents an odd parity value for the data value. For example, the value 0x03 (0011₂) will have a parity value of “1” since there are an even number of ones in the data value. In another example, the value 0x07 (0111₂) will have a parity value of “0,” since there are an odd number of ones in the value. The second parity bit may represent a column parity bit from the previous 6 packet set. The first column parity bit value may be for the most significant bit column of the previous 6 packet set. The next column parity bit may be for bit #4 of the previous 6 packet set, where the LSB is bit #0, and the most significant bit MSB is bit #5.

Table 8 of FIG. 14 illustrates the row parity and column parity values for the 18 sample example. As illustrated in Table. 8, the column parity bits for the first 6 data values (e.g., packet set A), is unknown since there was not a prior packet set to these data values. On the 7^{th} data value (e.g., Sample #7 in Table 8) sent from the remote IMD 16 to the hand held device 12, the column parity bit of the 7^{th} data value represents the MSB parity bit of the previous packet set. As an example, the MSB of each packet in the previous packet set is 101100₂. Accordingly, since there are an odd number of ones in this string of bits, the appropriate column parity bit is zero as illustrated in Table 8. The next column parity bit will be sent in the 8^{th} data packet between the IMD 16 and the hand held device 12, which represents bit #4 of the previous 6 packet set (e.g., the bit adjacent to the MSB). Accordingly, after the second 6 packet set is sent (e.g., packet set B), all the column parity bits for the first packet set (e.g., packet set A) is determined. In embodiments, the column parity for the last packet set (e.g., packet set C) may not be calculated.

The row parity bit and column parity bit provides the hand held device 12 the ability to correct single and multiple bit errors in packets sent from the IMD 16 using a dual parity scheme. In embodiments, the error detection module 406 (FIG. 4) detects errors in the packets sent from the IMD 16 to the hand held device 12. Additionally, according to some embodiments, the error correction module 408 can be configured to correct any detected errors in the packet sent from the IMD 16 to the hand held device 12.

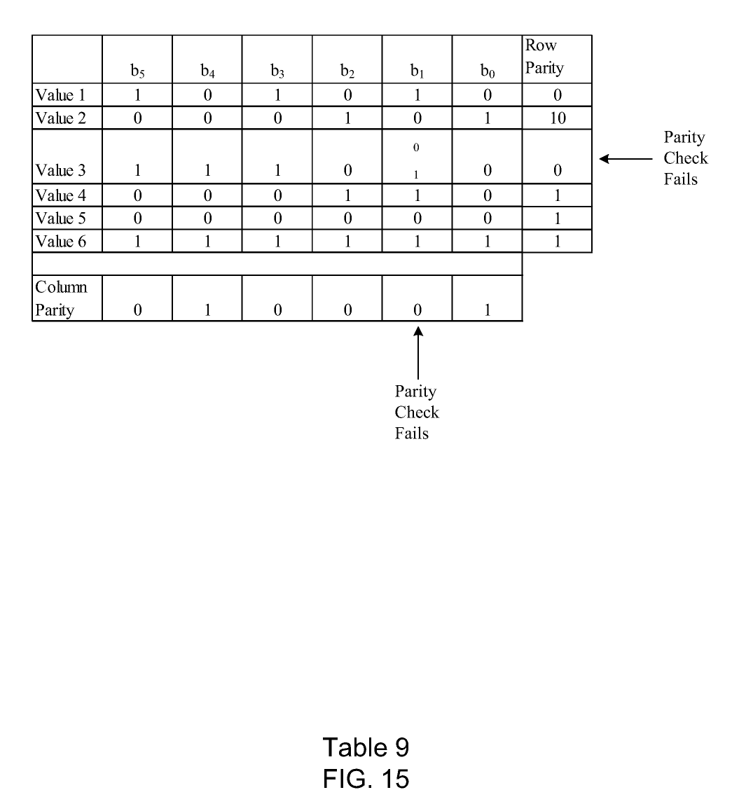

An example of a single bit error is shown in Table 9 of FIG. 15. In this example, bit #1 (e.g., b₁) of value #3 was supposed to be a zero, but was received as a 1. Accordingly, due to this error, there are an even number of ones in value #3. Additionally, because a one was transmitted instead of a zero, there are an even number of ones in the column representing bit #1. Thus, since the row parity bit is zero and the column parity bit is zero, there should be an odd number of ones for value #3 and an odd number of ones in the column representing bit #1. Thus, an error is detected by the error detection module 406 at bit #1 of value #3.

In embodiments, when the error detection module 406 detects an error, the error correction module 408 can be configured to correct the detected error. Referring to the previous example, the error correction module 408 can be configured to toggle or invert bit #1 on value #3, which corrects the error. Multiple errors within the same value can also be corrected.

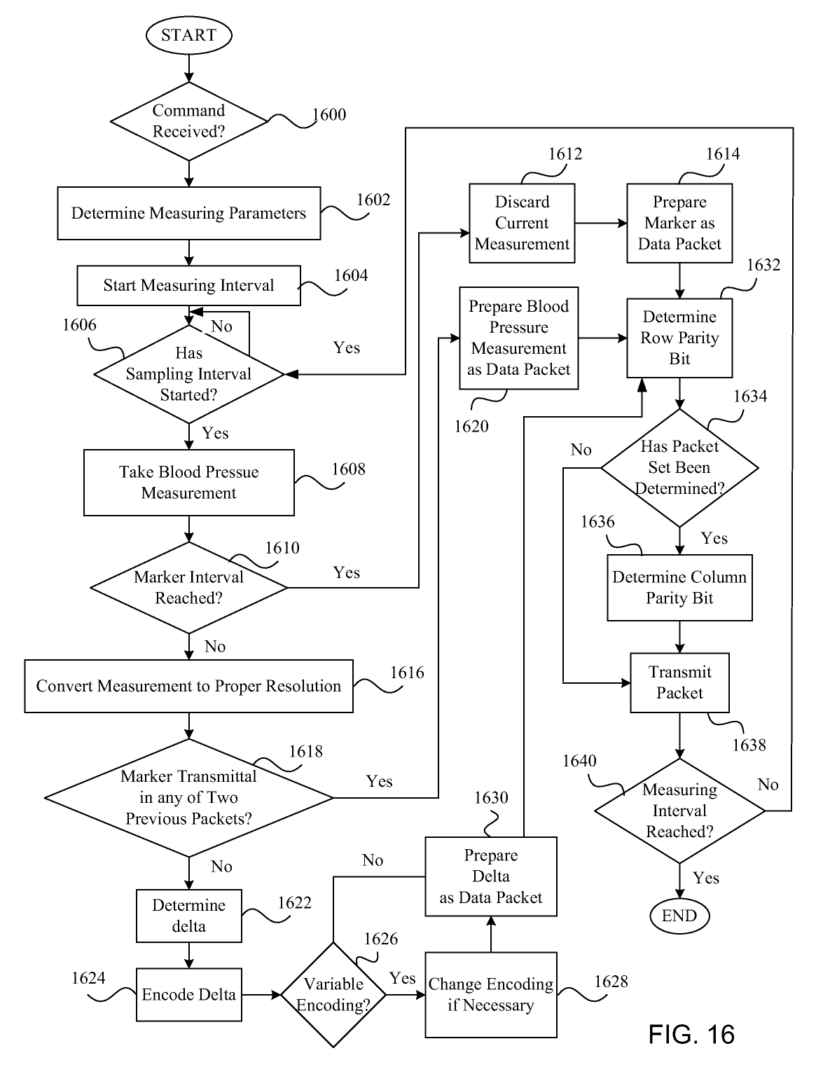

FIG. 16 illustrates an example process for sampling a blood pressure waveform and transmitting samples as data packets. In embodiments, the process illustrated in FIG. 16, may be implemented by the system 500 of FIG. 5 by the remote IMD 16 (FIG. 2). The process may generally start at 1600 where the response module 502 waits to receive a send data command from the send data command module 402. As an example, the response module waits for the command illustrated in Table 1 of FIG. 6. If the command is received, the process proceeds to 1602 where the response module 502 determines the measuring parameters. For example, using the send data command illustrated in Table 1, FIG. 6, the response module 502 determines that the measuring interval is 20 seconds, the sampling frequency is 40 Hz, the marker interval is 5 seconds, and the resolution is 0.5 mmhg. The process proceeds to 1604 where the pressure measurement module 504 starts sensing the blood pressure waveform 800 (FIG. 8) as described above.

The ADC module 506 waits for the sampling interval to start at 1606. If the sampling interval has started, the ADC module 506 takes a blood pressure sample at 1608. If it is time to send a marker at 1610, the packet module 514 discards the current sample at 1612. Referring to Table 8 of FIG. 14 as an example, it is illustrated that for sample #1, the sample measured by the ADC module 506 is discarded and replaced with the marker. The packet module 514 prepares the marker as a data packet at 1614. Preparing the marker as a data packet may include at least inserting a sync bit in front of the marker.

If the marker interval is not reached at 1610, the process proceeds to 1616 where the resolution conversion module 508 converts the sample taken by the ADC module 506 to the proper resolution as described above. If the marker was transmitted in any of the two previous packets at 518, the packet module 514 prepares the sample as the data packet 1620. As illustrated in Table 8 of FIG. 14, after the marker is sent, the next two values are the upper and lower six bits, respectively, of the base pressure measurement. Accordingly, at 1620, the sample prepared as a data packet includes 6 bits of the base pressure measurement. The first time the sample is prepared as a data packet at 1620, the upper 6 bits of the base pressure measurements are prepared as a data packet. The second time the sample is prepared as a data packet at 1620, the lower 6 bits of the base pressure measurement is prepared as a data packet.

If the marker was not transmitted in any of the previous two packets at 1618, the process proceeds to 1622 where the delta module 510 determines the delta 1622 as described above with respect to Table 7 of FIG. 13. The process proceeds to 1624 where the encoding module 512 encodes the delta using any desired encoding format. As an example, the encoding module 512 encodes the delta as described above with respect to Table 4 of FIG. 10.

If the system 500 is configured to perform variable encoding, the process proceeds from 1626 to 1628 where the encoding module 512 changes the encoding format, if necessary, as described above. If the system 500 is not configured to perform variable encoding, the process proceeds from 1626 to 1630 where the packet module 514 prepares the delta value as a data packet. Additionally, the process proceeds from 1628 to 1630 when the system 500 is configured to perform variable encoding. The packet module 1614 may prepare the delta value as a data packet by inserting at least the sync bit before the delta value. After the packet module has prepared a corresponding data packet at 1614, 1620, and 1630, the process proceeds to 1632 where the parity module 516 determines the row parity bit as described above, for example, with respect to Table 8 of FIG. 14. The process proceeds to 1634 to determine if the previous packet set has been determined. If the previous packet set has not been determined, the process proceeds to 1638 where the packet module 514 transmits the prepared data packet.

If the previous packet set has been determined, the process proceeds from 1634 to 1636 where the parity module 516 determines the column parity bit as described above with respect to Table 8 of FIG. 14. As an example, when the packet set A has not been completed, there is not enough data to determine a column parity bit. However, as illustrated in Table 8, once packet set A has been completed, the column parity bit is determined in packet set B for packet set A. After the column parity bit has been determined, the packet module 514 transmits the data packet at 1638. After the data packet has been transmitted by the packet module 514, the process ends at 1640 if the measuring interval has been reached. If the measuring interval has not been reached, the process returns to 1606 where the ADC module 506 waits for the next sampling interval to start.

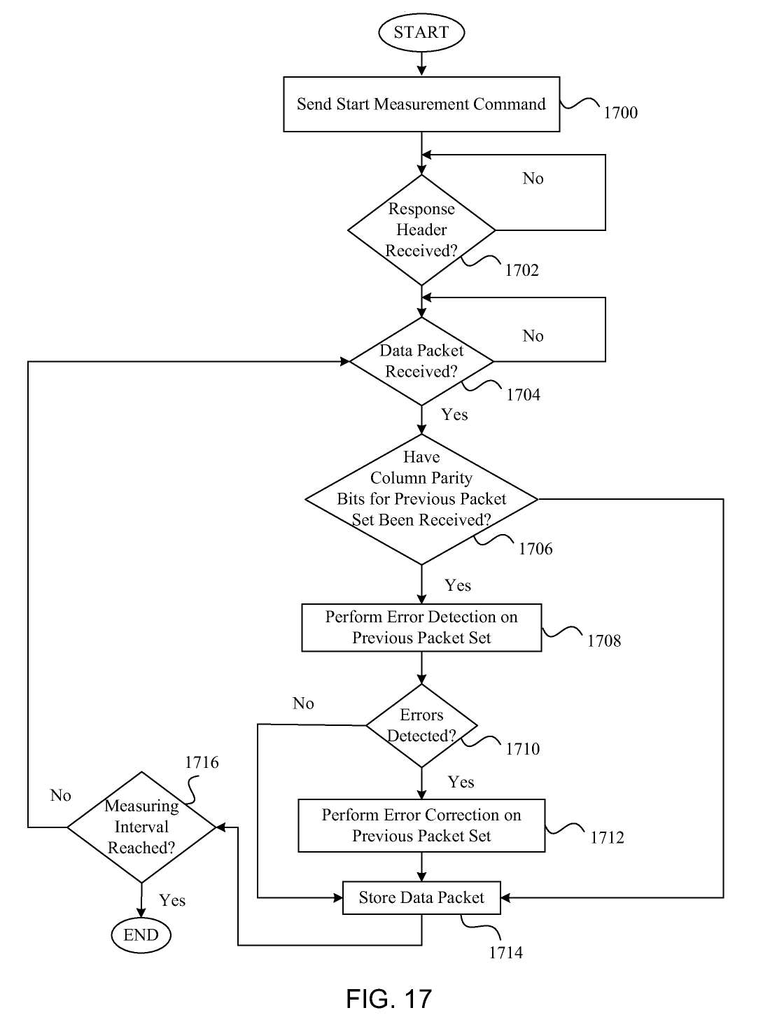

FIG. 17 illustrates an example process for receiving data packets and performing error detection and correction. In embodiments, the process illustrated in FIG. 17 is performed by the system 400 of FIG. 4 by the hand held device 12.

The process may generally start at 1700 where the send data command module 402 sends the send data command illustrated in Table 1 of FIG. 6. The process proceeds to 1702 where the read data module 402 waits to receive the response header. As an example, the read data module 402 waits for the response header illustrated in Table 2 of FIG. 7A. When the response header is received, the read data module 404 determines if a data packet has been received at 1704.

When the read data module 404 receives a data packet, the process proceeds to 1706 where the error detection module 406 determines if the column parity bits for the previous packet set have been received. If the column parity bits for the previous packet set have not been received, the process proceeds to 1714 where the read data module 404 stores the data packets. If the column parity bits for the previous packets that have been received, the process proceeds from 1706 to 1708 where the detection module 406 performs error detection on the previous packet set. As an example, as illustrated in Table 8 of FIG. 14, after the packet set B has been received, the error detection module 406 has received all the column parity bits for packet A.

Accordingly, at 1708, the error detection module 406 performs error detection on the previous packet set. The process proceeds to 1710 to determine if the error detection module 406 detected any errors. If the error detection module 406 did not detect any errors, the process proceeds to 1714 where the read data module 404 stores the data packet. If the error detection module detected errors, the process proceeds from 1710 to 1712 where the error correction module 408 performs error correction on the previous packet sets as described above with respect to Table 9 of FIG. 15. After the error correction has been performed, the read data module stores the data packet at 1714, which would be the corrected data packet in this scenario. The process proceeds from 1714 to 1716 where the read data module 404 determines if the measuring interval has been reached. If the measuring interval has not been reached, the process returns from 1716 to 1704 where the read data module waits for the next received data packet.

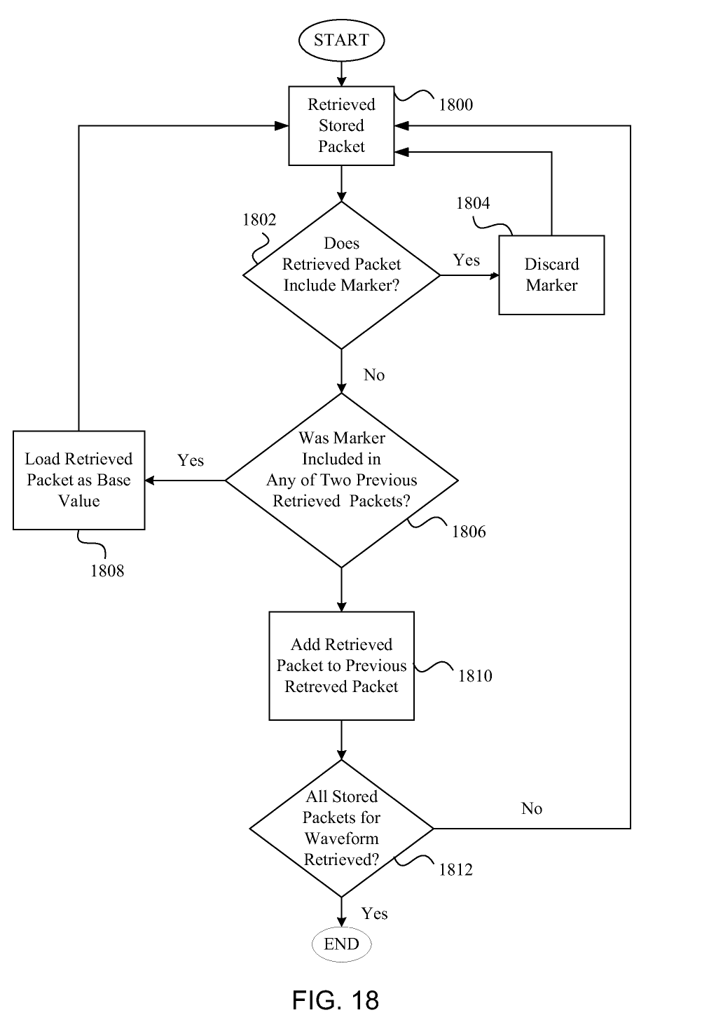

FIG. 18 illustrates an example process for reconstructing a waveform at the hand held device 12. In embodiments, the process illustrated in FIG. 18 is performed by the system 400.

The process may generally start at 1800 where the waveform reconstruction module 410 retrieves the stored data packet. If the retrieved packet includes the marker at 1802, the waveform reconstruction module 410 discards the marker at 1804 and returns to 1800 to receive the next stored data packet. If the retrieved data packet at 1802 does not include the marker, the waveform reconstruction module determines at 1806 if the marker was included in any of the two previously retrieved data packets. If the marker was included in any of the two previous retrieved data packets, the process proceeds from 1806 to 1808 where the waveform reconstruction module 410 loads the retrieved packet as a base value.

As illustrated in Table 8 of FIG. 14, after the marker is transmitted, the next two data packets include 6 bits of the base pressure measurement with the upper 6 bits of the base pressure measurement sent in the first packet after the marker, and the lower 6 bits of the base pressure measurement sent in the second packet after the marker is transmitted. If the marker was not included in any of the two previous retrieved packets, the process proceeds from 1806 to 1810 where the waveform reconstruction module 410 adds the retrieved packet to the previous retrieved packet. In this scenario, after the 12 bit base pressure measurement has been loaded at 1808, the waveform reconstruction module 410 starts adding the deltas to the base pressure measurement. The process proceeds from 1810 to 1812 to determine if all the stored packets for the blood pressure waveform have been retrieved. If all the stored packets have not been retrieved, the process returns to 1800. If all the stored packets have been retrieved, the process ends.

Embodiments of the present invention include various steps, which are described herein. The steps may be performed by hardware components or may be embodied in machine-executable instructions, which may be used to cause a general-purpose or special-purpose processor programmed with the instructions to perform the steps. Alternatively, the steps may be performed by a combination of hardware, software, firmware or human representatives of the parties or entities involved in the transaction.

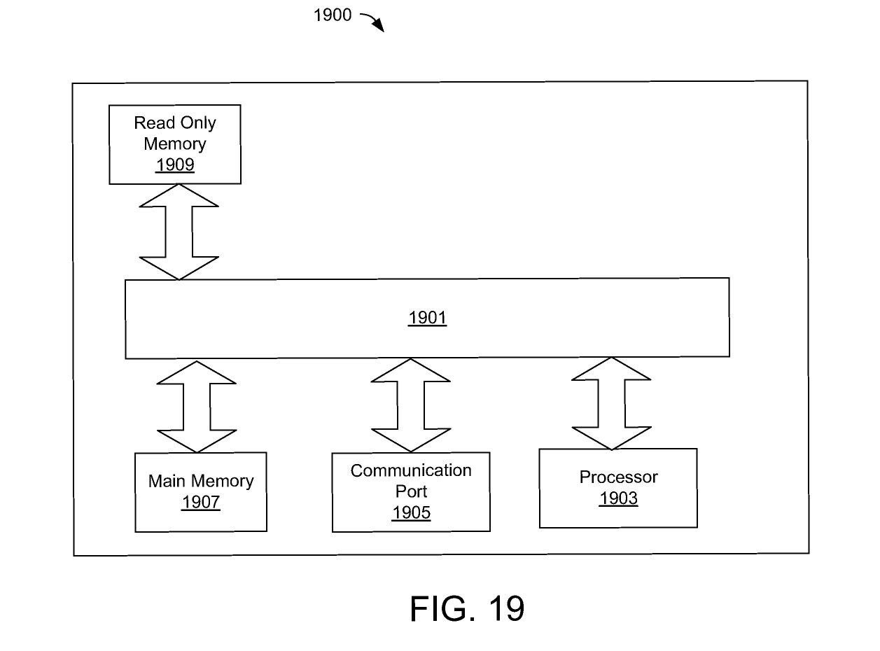

FIG. 19 is a schematic diagram of an example computing device 1900 upon which embodiments described herein may execute. The example computing device 1900 may be used to implement system 400 (FIG. 4) and/or system 500 (FIG. 5). The example computing device 1900 may be located on the IMD 16 (FIG. 2) and/or handheld device 12 (FIG. 3). One or more of the components illustrated in FIG. 19 may be used on the IMD 16 (FIG. 2) and/or the handheld device 12 (FIG. 3).

In embodiments, the computing device 1900 includes a bus 1901, at least one processor 1903, at least one communication port 1905, a main memory 1907, and a read only memory 1909. Processor(s) 1903 can be any known microcontroller, such as, but not limited to, a Motorola 68HC11, Intel 8051, Hitachi H8, or Texas Instruments MSP430.

Communication port(s) 1905 can be any known communications conduit such as, but not limited to, an RS-232 port for use with a modem based dialup connection, a 10/100 Ethernet port, or a Gigabit port using copper or fiber. Communication port(s) 1905 may be chosen depending on a network such as a Local Area Network (LAN), Wide Area Network (WAN), or any desired network to which the computing device 1900 connects. In alternative embodiments, the communication port(s) is configured to enable communication through any wireless communication channel such as an acoustic channel, RF channel, inductive channel, or infrared channel. The computing device 1900 may be in communication with peripheral devices such as, but not limited to, printers, speakers, cameras, microphones, or scanners.

Main memory 1907 can be Random Access Memory (RAM), or any other desired storage device(s). Read only memory 1909 (ROM) can be any suitable storage device(s) such as, but not limited to, an Erasable Programmable Read Only Memory (EPROM) chips for storing static information such as instructions for processor 1903.

In embodiments, the main memory 1907 and read only memory 1909 may each comprise of one or more units linked together physically or logically to operate as a single unit.

Bus 1901 communicatively couples processor(s) 1903 with the other memory, storage, and communication blocks. Bus 1901 can be a PCI/PCI-X or SCSI based system bus depending on the storage devices used.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method of processing blood pressure measurements transmitted from an implantable medical device including a blood pressure sensor to a receiving device, the method comprising:
transmitting a command to the implantable medical device, the command prompting the implantable medical device to sense blood pressure measurements;
receiving a response from the implantable medical device, the response indicating that the device initiated the sensing of blood pressure measurements;
receiving one or more data packets from the implantable medical device, the one or more data packets including:
a base pressure measurement representative of a starting point of a blood pressure waveform, and
a measurement representative of a difference between another blood pressure measurement and the base pressure measurement; and
generating a pressure waveform from the one or more packets.

2. The method according to claim 1, wherein the command includes at least a measuring interval indicating a time interval for sampling the blood pressure waveform, a marker interval indicating a time interval for sending a marker, and a sampling rate indicating a time interval between sampling sensed blood pressure measurements of the blood pressure waveform.

3. The method according to claim 1, further comprising:
performing error correction on the one or more received packets using one or more parity bits included in each packet of the received one or more data packets, wherein
the one or more parity bits included in each packet of the received one or more data packets includes at least one row parity bit and at least one column parity bit; and
detecting an error when at least one bit in the one or more received packets does not correspond to the at least one row parity bit and the at least one column parity bit.

4. The method according to claim 1, further comprising:
synchronizing the one or more received packets using a marker included in the one or more received packets, the marker having a unique value non-representative of the blood pressure measurements, wherein
a first packet received after the marker is received is identified as a first portion of the base pressure measurement, and
a second packet received after the first packet is identified as a second portion of the base pressure measurement.

5. The method according to claim 1, further comprising:
reconstructing a blood pressure waveform from the received one or more data packets, wherein, for each data point on the blood pressure waveform, the measurement representative of a difference between the another blood pressure measurement and the base pressure measurement is added to the base pressure measurement.

6. The method according to claim 1, wherein the measurement representative of the difference is encoded using an encoding range including a range of discrete values.

7. The method according to claim 1, wherein the one or more data packets include at least a sync bit, a predetermined number of data bits, a row parity bit, and a column parity bit.

8. An implantable medical device configured to sample a blood pressure waveform, the device comprising:
a response module configured to receive a command from a communicating device, the command including parameters for sampling a blood pressure waveform, the parameters specifying at least a sampling rate and a resolution;
the response module configured to transmit a response packet to the communicating device in response to the command;
a pressure measurement module configured to sense the blood pressure waveform;
an ADC module configured to obtain a blood pressure sample by sampling the blood pressure waveform at the sampling rate;
a resolution conversion module configured to convert the blood pressure sample to the resolution;
a delta module configured to determine a difference measurement between the converted blood pressure sample and a previous blood pressure sample;
an encoding module configured to encode the determined difference measurement using an encoding range including a range of discrete values;
a packet module configured to generate a data packet including the difference measurement; and
the packet module configured to transmit the data packet to the communicating device.

9. The device of claim 8, wherein:
the packet module is further configured to transmit a marker to the device in accordance with a marker interval, the marker interval included in the command, the marker having a unique value non-representative of the blood pressure measurement;
the packet module is further configured to transmit a first packet after transmission of the marker, the first packet including a first portion of a base pressure measurement; and
the packet module is further configured to transmit a second packet after transmission of the first packet, the second packet including a second portion of the base pressure measurement.

10. The device of claim 8, further comprising:
a parity module configured to determine a row parity bit by counting a number of ones included in the data packet; and
the parity module configured to determine a column parity bit of a packet set transmitted prior to the transmission of the data packet,
wherein the packet module is further configured to prepare the data packet by inserting the row parity bit and column parity bit in the data packet.

11. The device of claim 8, wherein the encoding module is further configured to adjust the encoding range using a range of the blood pressure values of the sampled blood pressure waveform.

12. The device of claim 8, wherein:
the encoding module is further configured to determine an overflow result when the difference measurement exceeds a maximum range of the encoded range; and
the packet module is further configured to transmit the overflow result in another data packet.

13. The device of claim 8, wherein the data packet includes at least a sync bit, a number of data bits, a row parity bit, and a column parity bit.

14. A system for sampling a blood pressure waveform, the system comprising:
an external device communicable with an implantable medical device, the external device including:
a command module configured to transmit a command to the implantable medical device, the command including parameters for sampling a blood pressure waveform; and
the implantable medical device including:
a response module configured to transmit a response packet to a second device in response to the command;
a pressure measurement module configured to obtain a blood pressure measurement by sampling the blood pressure waveform at a sampling rate;
a resolution conversion module configured to convert the blood pressure measurement to a resolution;
a delta module configured to determine a difference measurement between the converted sampled blood pressure waveform and a previous sample;
an encoding module configured to encode the determined difference measurement using an encoding range including a range of discrete values; and
a packet module configured to prepare the determined difference measurement as a data packet;
wherein the packet module is configured to transmit the data packet to the external device.

15. The system according to claim 14, wherein:
the packet module is further configured to transmit a marker to the external device in accordance with a marker interval, the marker interval included in the command, the marker having a unique value non-representative of the blood pressure measurement;
the packet module is further configured to transmit a first packet after transmission of the marker, the first packet including a first portion of a base pressure measurement; and
the packet module is further configured to transmit a second packet after transmission of the marker, the second packet including a second portion of the base pressure measurement.

16. The system according to claim 14, wherein the implantable device further comprises:
a parity module configured to determine a row parity bit by counting a number of ones included in the data packet;
the parity module is configured to determine a column parity bit of a packet set transmitted prior to the transmission of the data packet; and wherein
the packet module is further configured to prepare the data packet by inserting the row parity bit and column parity bit in the data packet.

17. The system according to claim 14, wherein the external device further includes:
an error detection module configured to detect one or more errors in the data packet using at least the row parity bit and the column parity bit; and
an error correction module configured to correct the one or more errors in the data packet.

18. The system according to claim 14, wherein
the encoding module is further configured to adjust the encoding range using a range of the sampled blood pressure waveform.

19. The system according to claim 14, wherein the packet module is further configured to determine an overflow result when the determined difference exceeds a maximum range of the encoded range and transmit the overflow result in another data packet.

20. The system according to claim 14, wherein the external device further includes:
a waveform reconstruction module configured to reconstruct the blood pressure waveform by adding the encoded difference included in the data packet to a base pressure measurement.
